(19) United States
(12) Patent Application Publication
(10) Pub. No.: US 2004/0170598 A1
(43) Pub. Date: Sep. 2, 2004

(54) Polysubstituted polycarboxylic phosphoamide biopolymers, methods for their production and uses of compositions derived therefrom

(76) Inventor: Emilio Barragan, Benito Juarez (MX)

Correspondence Address: Doran R Pace, Saliwanchik Lloyd & Saliwanchik, Professional Association, Suite A-1 2421 N W 41st Street, Gainesville, FL, 32606-6669, US

(21) Appl. No.: 10/473,928

(22) PCT Filed: Mar. 4, 2002
(86) PCT No.: PCT/IB02/00655

(30) Foreign Application Priority Data
Apr. 5, 2001 (CA) 2,343,548

(51) Int. Cl. A61K 38/17; C08G 63/48; C08G 63/91

(52) U.S. Cl. 424/78.27; 525/54.1

(57) ABSTRACT

The present invention provides polysubstituted polycarboxylic phosphoamide biopolymers (PPPBs) comprising polysaccharides and glycopolypeptides attached to a phospho-citrate backbone. Phosphorylation of the biopolymers yields phosphorylated polysubstituted polycarboxylic phosphoamide biopolymers (pPPPBs) which can be used as inflammatory response modulators, immunomodulators and/or biological response modifiers. Methods for producing the PPPBs in yeast subjected to multiple chemical stressors, and uses of compositions derived therefrom, are provided.

### THE FIELD OF THE INVENTION

The present invention relates to bioactive biopolymers produced in yeast and their use as immunomodulators.

### THE BACKGROUND OF THE INVENTION

There is great interest in the use of therapeutic materials which can enhance the response of the immune system to facilitate healing in a number of different patients, including cancer patients, particularly those undergoing radiation therapy or chemotherapy, chronic drug users, critically ill patients, such as those with severe bums or complications of sepsis or of multiple trauma, people suffering from chronic severe stress, bum patients, patients who receive exogenous adrenal corticosteroids or synthetic analogs for extended periods of time, to control diseases such as cancer, as well as diseases caused by external agents such as viruses or bacteria. Although many effective immune system modulators exist, the majority of these have moderate to severe toxic side effects.

Wound healing requires a coordinated influx of fibroblasts, vascular endothelium and epithelium. There is clearly a recognized need in the art for new agents and methods which promote wound healing. Agents useful in treating wound healing can be identified and tested in a number of in vitro and in vivo models.

When an injury occurs, cell damage comes from the precipitating event, for example a cut or bum, resulting in ruptured cells and severed or crushed capillaries and other blood vessels. The interruption of blood flow produces anoxia, causing the death of additional cells. Within 15 minutes of injury the wound is filled with dead and dying cells, extracellular substances (collagen, elastic fibers, fat and ground substances), extravasated blood, and possibly bacteria and viruses introduced by the injurious agent. Tissue damage is not restricted to the initial area of injury. It may increase over the next several hours or days as a result of the release of lysomal enzymes from the injured cells or as a consequence of swelling and infection.

Typical wound healing takes anywhere from 5 to 21 days. This time period is of course longer for the immune compromised patient because such patients are frequently unable to sufficiently stabilize the wound and ward off infection which prevents the proper adherence of fibrin, fibronectin or collagen at an acceptable rate at the locus of the wound. For example, those with vasculitis or other rheumatic or diabetic diseases frequently experience wound healing times far in excess of several weeks. Diabetics frequently develop lesions that take weeks to heal. Others, such as those with artificial limbs have continuous injury at the point of contact between the limb and the point of attachment to the body. Burns also present healing problems insofar as the burned tissue is incapable of timely production of fibrin. Accordingly, there is a great need to shorten the duration of time necessary for wound or burn healing to occur.

Some naturally occurring biopolymers have been developed as immune system modulators. Fermentation of bacteria has been used to prepare pharmacologically active nitrogenated polysaccharides (FR2582672). U.S. Pat. No. 5,766,894 describes the production of vitamin B₆ by fermentation of Rhizobium. U.S. Pat. No. 4,975,421,4,900,722, 4,877,777, 4,833,131, 4,818,752, and 4,761,402, describe a soluble phosphorylated glucan derived from the yeast Saccharomyces cerevisiae for therapeutic use. European Patent Application Nos. EP0491114 and EP0511932 describe a soluble biopolymer isolated from dead yeast having pharmacological activity.

Polysaccharides, (carbohydrate polymers in which the repeating units or building blocks are sugars) are one example of a biopolymer that has been produced and extracted from yeast for use as therapeutics and immunomodulators. A variety of naturally occurring homopolysaccharides or polyglucoses, including polymers such as cellulose, amylose, glycogen, laminarians and starch are referred to generically as glucans.

One notable example of polyglucose immunomodulators are the β-glucans which have profound effects on both the reticuloendothelial and immune systems. Previous studies have demonstrated that in vivo administration of particulate glucan to a variety of experimental animals induces a number of profound immunobiological responses, including the following: (1) enhanced proliferation of monocytes and macrophages (Deimann and Fahimi (1979) J. Exper. Med. 149:883-897; Ashworth et al. (1963) Expt. Molec. Pathol., Supp. 1:83-103); (2) enhanced macrophage phagocytic function (Riggi and Di Luzio (1961) Am. J. Physiol. 200: 297-300): (3) enhanced macrophage secretory activity (Barlin et al. (1981) in Heterogeneity of Mononuclear Phagocytes, Forster and Landy, eds., Academic Press, New York, pp.243-252); (4) increased macrophage size (Patchen and Lotzova (1980) Expt. Hematol. 8:409-422); (5) enhanced macrophage adherence and chemotactic activity (Niskanen et al. (1978) Cancer Res. 38:1406-1409); and (6) enhanced complement activation (Glovsky et al. (1983) J. Reticuloendothel. Soc. 33:401-413). Increased cytolytic activity against tumor cells has been demonstrated in macrophages from animals and man treated with particulate glucan both in vivo (Mansell and Di Luzio (1976) in The Macrophage in Neoplasia, Academic Press, New York, pp. 227-243) and in vitro (Chirigos et al. (1978), Cancer Res. 38:1085-1091).

In addition to effects on reticuloendothelial and immune responses, in vivo administration of particulate glucan has been demonstrated to enhance hemopoietic8 activity including granulopoiesis, monocytopoiesis and erythropoiesis leading to greater recovery from a lethal dose of whole body irradiation (Patchen (1983) Surv. Immunol. Res. 2:237-242). A number of studies have indicated that in vivo administration of particulate glucan significantly modifies host resistance to a wide variety of infectious diseases induced by bacterial, fungal, viral and parasitic organisms. (Di Luzio (1983) Trends in Pharmacol. Sci. 4:344-347). Extensive studies have indicated that particulate glucan has potent anti-cancer activity (Di Luzio et al. (1979) in Advances in Experimental Medicine and Biology, Vol. 121A: 269-290; Williams et al. (1985) Hepatology 5:198-206).

Particulate glucan-induced macrophage activation has also been implicated in promoting of wound healing (Mansell and DiLuzio (1976), in The Macrophage in Neoplasia, Academic Press, New York, pp. 227-243). Israel and Edelstein, 1978, in “Immune Modulation and Control of Neoplasia,” Chirigos, ed., Raven Press, New York, pp. 255-280). Wound healing consists of a series of processes whereby injured tissue is repaired, specialized tissue is regenerated, and new tissue is reorganized. Wound healing consists of three major phases: a) an inflammation phase (0-3 days), b) a cellular proliferation phase (3-12 days), and (c) a remodeling phase (3 days-6 months). During the inflammation phase, platelet aggregation and clotting form a matrix which traps plasma proteins and blood cells to induce the influx of various types of cells. During the cellular proliferation phase, new connective or granulation tissue and blood vessels are formed. During the remodeling phase, granulation tissue is replaced by a network of collagen and elastin fibers leading to the formation of scar tissue. Thus, topical administration of particulate glucan resulted in the activation and recruitment of macrophages to the wound area, which subsequently enhanced proliferation of fibroblasts and capillaries culminating in accelerated healing of the wound.

This background information is provided for the purpose of making known information believed by the applicant to be of possible relevance to the present invention. No admission is necessarily intended, nor should be construed, that any of the preceding information constitutes prior art against the present invention.

### THE SUMMARY OF THE INVENTION

It is, therefore, an object of the present invention to provide polysubstituted polycarboxylic phosphoamide biopolymers (PPPBs). Phosphorylation of the biopolymers yields phosphorylated polysubstituted polycarboxylic phosphoamide biopolymers (pPPPB) which can be used as inflammatory response modulators, immunomodulators and/or biological response modifiers to facilitate healing in a wide variety of diseases or disorders, including physical wounds and bums. Methods for producing the PPPBs in yeast that have been subjected to multiple chemical stressors, and uses of pPPPBs derived therefrom, are also provided.

In accordance with one aspect of the present invention, there is provided a biopolymer as in Formula 1.

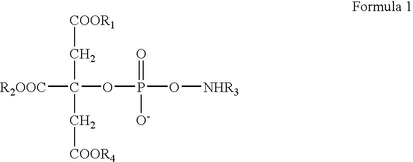

wherein R₁, R₂, R₃ and R₄ are selected from the group comprising a hydrogen, a glycopolypeptide, a polysaccharide, a branched glycopolypeptide, and a branched polysaccharide, wherein the biopolymer comprises up to four glycopolypeptides in total, four polysaccharides in total, or any combination of glycopolypeptides and/or polysaccharides totaling four, and wherein the biopolymer must contain at least one glycopolypeptide or at least one polysaccharide moiety.

Once phosphorylated, the derivative biopolymers of Formula 1 are as depicted in Formula 2.

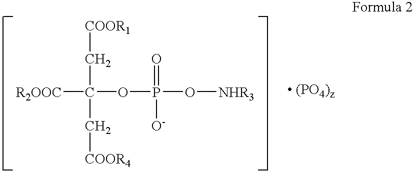

wherein, R₁, R₂, R₃ and R₄ are selected from the group comprising a hydrogen, a glycopolypeptide, a polysaccharide, a branched glycopolypeptide, and a branched polysaccharide, wherein the biopolymer comprises up to four glycopolypeptides in total, four polysaccharides in total, or any combination of glycopolypeptides and polysaccharides totaling four, and wherein the biopolymer contains at least one glycopolypeptide or at least one polysaccharide moiety, and wherein z indicates a ratio of phosphate groups to biopolymer such that the weight of the phosphate groups constitutes less than or equal to 3% of the total weight of the compound according to Formula 2.

The foregoing objects are achieved by a method of producing PPPBs comprising the steps of sequentially: (a) cultivating a strain of yeast cells to produce a standard stock culture; (b) stressing a portion of said standard stock culture using an initial concentration of a first stressor molecule to produce a modified stock culture comprising yeast cells that can survive in the presence of the initial concentration of the first stressor molecule; (c) repeating step (b) at least once using the modified stock culture in place of the standard stock culture and using a stressor molecule that is the same or different from the first stressor molecule; (d) cultivating a portion of the modified stock culture produced in step (c) in the presence of the stressor molecules to generate a production culture; (e) isolating the PPPBs from said production culture; and (f) phosphorylating the PPPBs to produce pPPPBs.

Various other objects and advantages of the present invention will become apparent from the detailed description of the invention.

### BRIEF DESCRIPTION OF THE FIGURES AND TABLES

FIG. 1 presents a graph showing a standard growth curve for yeast cells of the genus Candida. The abscissa shows the time of growth in days and the ordinate the number of yeast grown in logarithmic units.

FIG. 2 shows a UV absorption spectrum for the pPPPBs. A broad band centered at 196 nm corresponds to the maximum UV absorption for the pPPPBs. The abscissa corresponds to UV absorption in units of mn.

FIG. 3 shows an HPLC analysis (A) showing protein content of the pPPPBs determined following its derivatization with 4-dimethylaminoazobenzene-4′-sulfonyl chloride (dabsyl chloride), and an HPLC analysis (B) showing protein content of a standard, albumin, determined following its derivatization with dabsyl chloride.

FIG. 4 presents a trace showing the ¹H-NMR analysis of the pPPPBs. A peak at 3.5 ppm indicates the presence of carboxyl groups. Peaks at 2.580, 2.550, 2.446 and 2.415 indicate the presence of methylene groups.

FIG. 5 presents a trace showing the ¹³C-NMR analysis of the pPPPBs. A peak at 76.156 ppm indicates the presence of a phosphoamide and a peak at 46.695 ppm indicates the presence of carboxyl groups.

FIG. 6 presents results of a demonstration of Phagocytic Index in patients with a compound fracture of a long bone following treatment with either the pPPPBs (light bars) or placebo (dark bars). The abscissa represents the number of yeast cells engulfed by phagocytic cells in a patient's blood sample. Data are organized in the following phagocytosis categories: phagocytic cells containing zero yeast cells, phagocytic cells containing 1 to 2 yeast cells, phagocytic cells containing 3 to 5 yeast cells, phagocytic cells containing 6 to 9 yeast cells and phagocytic cells containing >10 yeast cells. The ordinate represents the percentage, from a total of 100%, of yeast cells engulfed within all phagocytosis categories.

FIG. 7 presents results of a demonstration of Phagocytic Index in patients with wounds to the abdomen and/or thorax following treatment with either the pPPPBs (light bars) or placebo (dark bars). Values for the phagocytic index were determined as described for FIG. 6.

FIG. 8 presents results of a study demonstrating Phagocytic Index in patients treated for grade II or III head concussions following treatment with either the pPPPBs (light bars) or placebo (dark bars). Values for the phagocytic index were determined as described for FIG. 6.

FIG. 9 demonstrates an effect of the pPPPBs on the number of leukocytes/ml of blood drawn from trauma patients upon arrival and discharge from hospital. The abscissa represents the two time periods upon which blood samples were withdrawn from patients, arrival and discharge. The ordinate represents the number of leukocytes/ml of blood drawn from patients under each condition given either the pPPPBs or placebo. Symbols on the figure represent a) patients presenting with head concussions and treated with the pPPPBs, ♦-♦; b) patients presenting with head concussions and treated with placebo, ▪-▪; c) patients presenting with a compound fracture of a long bone and treated with the pPPPBs, ▴-▴; d) patients presenting with a compound fracture of a long bone and treated with placebo, x-x; e) patients presenting with a wound to the abdomen and/or thorax and treated with the pPPPBs, *-*; and f) patients presenting with a wound to the abdomen and/or thorax and treated with placebo, ●-●.

FIG. 10 demonstrates effects of the pPPPBs on the number of platelets/ml of blood drawn from trauma patients upon arrival and discharge from hospital. The abscissa represents the two time periods upon which blood samples were withdrawn from patients, arrival and discharge. The ordinate represents the number of platelets/ml of blood drawn from patients under each condition given either pPPPBs or placebo. Symbols on the figure represent a) patients presenting with head concussions and treated with the pPPPBs, ♦-♦; b) patients presenting with head concussions and treated with placebo, ▪-▪; c) patients presenting with a compound fracture of a long bone and treated with the pPPPBs, ▴-▴; d) patients presenting with a compound fracture of a long bone and treated with placebo, x-x; e) patients presenting with a wound to the abdomen and/or thorax and treated with the pPPPBs, *-*; and f) patients presenting with a wound to the abdomen and/or thorax and treated with placebo, ●-●.

FIG. 11 presents effects of the pPPPBs on the length of time spent in hospital by patients. Bars number 1 and 2 represent patients treated for head concussion given either the pPPPBs (bar 1) or placebo (bar 2), respectively. Bars number 3 and 4 represent patients treated for a compound fracture of a long bone given either the pPPPBs (bar 3) or placebo (bar 4), respectively. Bars number 5 and 6 represent patients treated for wounds to the abdomen and/or thorax given either the pPPPBs (bar 5) or placebo (bar 6), respectively.

FIG. 12 presents a trace showing the ³¹P-NMR analysis of the pPPPBs. A peak at 3.021 indicates a phosphate group.

FIG. 13 presents traces showing the IR spectra for the purified pPPPBs (A) and the isolated pPPPBs (B), respectively.

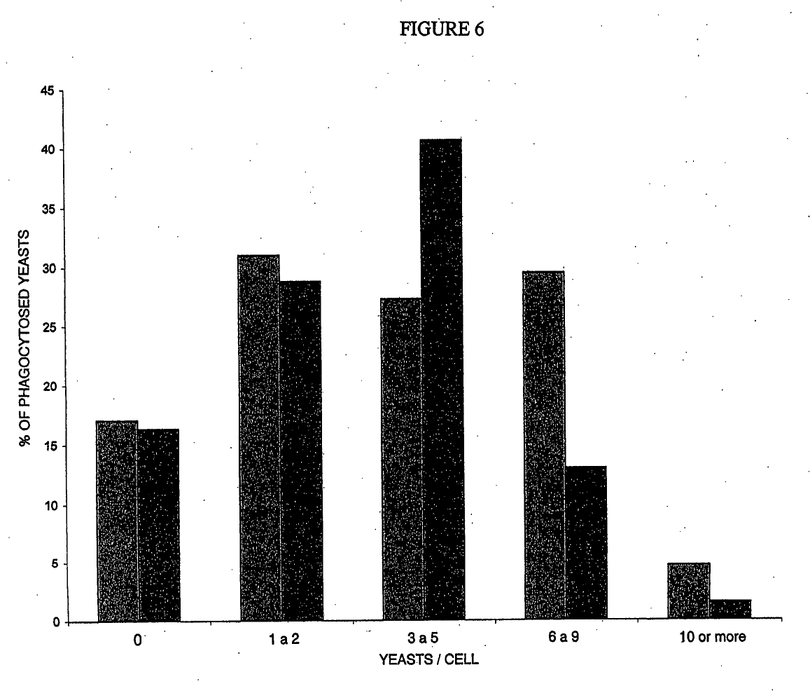

Table 1 shows results of a study demonstrating a Phagocytic Index in patients presenting with grade II or III head concussions and treated with either pPPPBs or placebo. Yeast/Cells represents the number of yeast cells engulfed by phagocytic cells in a patients blood sample, see detailed description for further details. Data are organized as described for FIG. 6.

Table 2. A table showing the effect of the pPPPBs on hematocrit percentage (Hto(%)), percent hemoglobin in the blood (Hb(%)), number of leukocytes/ml of blood drawn and the number of platelets×10³/ml of blood drawn in patients presenting with grade II or III head concussions upon their admission to, and release from, hospital. “Number” designates the number given to the patient.

Table 3 shows results of a study demonstrating the effect of placebo on hematocrit percentage (Hto(%)), percent hemoglobin in the blood (Hb(%)), number of leukocytes/ml of blood drawn and the number of platelets×10³/ml of blood drawn in patients presenting with grade II or III head concussions upon their admission to, and release from, hospital. “Number” designates the number given to the patient.

Table 4 shows results of a study demonstrating Phagocytic Index for patients treated for a compound fracture of the long bones following treatment with either the pPPPBs or placebo. Yeast/Cells represents the number of yeast cells engulfed by phagocytic cells in a patients blood sample, see detailed description for further details. Data are organized as described for FIG. 6.

Table 5 shows results of a study demonstrating an effect of the pPPPBs on hematocrit percentage (Hto(%)), percent hemoglobin in the blood (Hb(%)), number of leukocytes/ml of blood drawn and the number of platelets×10³/ml of blood drawn in patients presenting with a compound fracture of the long bones upon their admission to, and release from, hospital. “Number” designates the number given to the patient.

Table 6 shows results of a study demonstrating an effect of placebo on hematocrit percentage (Hto(%)), percent hemoglobin in the blood (Hb(%)), number of leukocytes/ml of blood drawn and the number of platelets×10³/ml of blood drawn in patients presenting with a compound fracture of the long bones upon their admission to, and release from, hospital. “Number” designates the number given to the patient.

Table 7 shows results of a study demonstrating a Phagocytic Index for patients presenting with a penetrating wound to the abdomen and/or thorax following treatment with either the pPPPBs or placebo. Yeast/Cells represents the number of yeast cells engulfed by phagocytic cells in a patients blood sample, see detailed description for further details. Data are organized as described for FIG. 6.

Table 8 shows results of a study demonstrating an effect of the pPPPBs on hematocrit percentage (Hto(%)), percent hemoglobin in the blood (Hb(%)), number of leukocytes/ml of blood drawn and the number of platelets×10³/ml of blood drawn in patients presenting with a penetrating wound to the abdomen and/or thorax upon their admission to, and release from, hospital. “Number” designates the number given to the patient.

Table 9 shows results of a study demonstrating an effect of placebo on hematocrit percentage (Hto(%)), percent hemoglobin in the blood (Hb(%)), number of leukocytes/ml of blood drawn and the number of platelets×10³/ml of blood drawn in patients presenting with a penetrating wound to the abdomen and/or thorax upon their admission to, and release from, hospital. “Number” designates the number given to the patient.

Table 10 shows results of a study demonstrating an effect of the pPPPBs or placebo on the length of time spent in hospital by patients treated for head concussion.

Table 11 presents results of a study demonstrating an effect of pPPPBs or placebo on the length of time spent in hospital by patients treated for a compound fracture of the long bones.

Table 12 shows results of a study demonstrating an effect of the pPPPBs or placebo on the length of time spent in hospital by patients treated for a wound to the abdomen and/or thorax.

Table 13 describes chemical and biochemical characterization of the phosphorylated biological response modifier pPPPBs, purified and mixed with calcium salts.

Table 14 characterizes the range of hematological parameters following zero (G0), one (G1), two (G2), three (G3) or four (G4) chemotherapy sessions in patients with cancer. Cancers included: ovarian, breast, lymphatic, rectal, colon, stomach, lung, kidney, cervical, bone as well as abdominal and sinovial sarcomas.

Table 15 characterizes the average hematological parameters following four (G4) chemotherapy sessions which included treatment with pPPPBs in patients with cancer. Cancers included: ovarian, breast, lymphatic, rectal, colon, stomach, lung, kidney, cervical, bone as well as abdominal and sinovial sarcomas.

Table 16 presents a characterization of the average hematological parameters in cancer patients following radical surgery procedures and treatment with pPPPBs before, during and following surgery. Patients received no chemotherapy or radiation therapy prior to, or following, surgery.

### DETAILED DESCRIPTION OF THE INVENTION

This invention provides polysubstituted polycarboxylic phosphoamide biopolymers (PPPBs). Phosphorylation of the biopolymers yields phosphorylated polysubstituted polycarboxylic phosphoamide biopolymers (PPPPB) which can be used as inflammatory response modulators, immunomodulators and/or biological response modifiers to facilitate healing in a wide variety of diseases or disorders, including physical wounds and bums. Methods for producing the PPPBs in yeast, derivatizing them to produce pPPPBs, and uses of compositions derived therefrom, are provided.

Structure of Polysubstituted Polycarboxylic Phosphoamide Biopolymers

The present invention provides PPPBs of Formula 1:

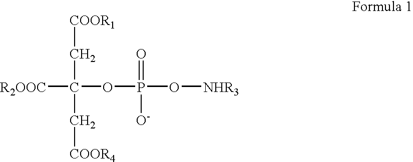

wherein R₁, R₂, R₃ and R₄ are selected from the group comprising a hydrogen, a glycopolypeptide, a polysaccharide, a branched glycopolypeptide, and a branched polysaccharide, wherein the biopolymer comprises up to four glycopolypeptides in total, four polysaccharides in total, or any combination of glycopolypeptides and/or polysaccharides totaling four, and wherein the biopolymer must contain at least one glycopolypeptide or at least one polysaccharide moiety.

The glycopolypeptides may range in size and make up no less than 0.1% or no more than 0.5% of the total weight of the biopolymer. In one embodiment of the present invention the size of glycopeptides ranges from about 14 to 16 kDa. In addition, the polysaccharide content of the biopolymer ranges from about 0.1% to about 0.9% of the total weight.

The following structures are provided as examples to highlight the types of possible configurations of the structures encompassed within the scope of Formula 1. It should be understood that these examples are provided for illustrative purposes only. Therefore, they should not limit or restrict the scope of this invention in any way or the number of possible structures included within Formula 1.

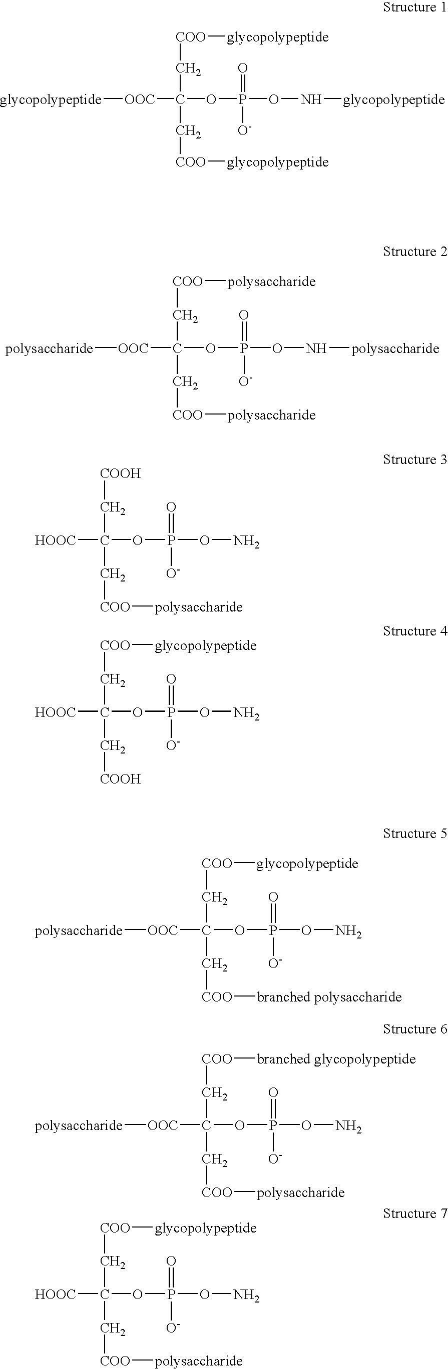

An alternate means of describing the biopolymers of this invention is depicted in Formula 1A.

[(C₆H₅O₁₀NP)-(glycopolypeptide)_{X}-(polysaccharide)_{Y}] Formula 1A

wherein, x equals 0-4; y equals 0-4, and wherein the sum of x plus y is less than or equal to 4 and greater than or equal to 1.

The present invention further provides phosphorylated derivatives of PPPBs, which are referred to as pPPPBs and are depicted in Formula 2

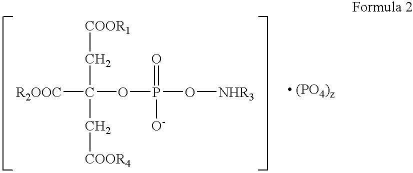

wherein R₁, R₂, R₃ and R₄ are selected from the group comprising a hydrogen, a glycopolypeptide, a polysaccharide, a branched glycopolypeptide, and a branched polysaccharide, wherein the biopolymer comprises up to four glycopolypeptides in total, four polysaccharides in total, or any combination of glycopolypeptides and/or polysaccharides totaling four, and wherein the biopolymer must contain at least one glycopolypeptide or at least one polysaccharide moiety, and wherein z indicates a ratio of phosphate groups to biopolymer such that the weight of the phosphate groups constitutes less than or equal to 3% of the total weight of Formula 2.

An alternative means of describing the pPPPBs is depicted in Formula 2A

[(C₆H₅O₁₀NP)-(glycopolypeptide)_{X}-(polysaccharide)_{Y}]-[(PO₄)_{z}] Formula 2A

wherein, x equals 0-4; y equals 0-4; and z indicates a ratio of phosphate groups to biopolymer such that the weight of the phosphate groups constitutes less than or equal to 3% of the total weight of Formula 2A; and wherein the sum of x plus y is less than or equal to 4 and greater than or equal to 1.

Preparation of Polysubstituted Polycarboxylic Phosphoamide Biopolymers

One method of preparing PPPBs comprises the following sequential steps: 1) preparing a strain of yeast; 2) using a fermentation process familiar to someone skilled in the art to cultivate said strain of yeast under sequential additions of stressors to the yeast cells and selecting for live (successful) strains; 3) adding a polycarboxylic acid to provide a backbone for PPPBs; and 4) producing conditions which increase the phosphorylation state of the purified compound to make pPPPBs.

In one embodiment of the present invention, the polycarboxylic acid which is used to provide a backbone for PPPB's can be an hydroxypolycarboxylic acid, for example citric acid or tartaric acid. Prior to fermentation, a polycarboxylic acid is treated with a phosphorylating agent, for example phosphoric acid, and an agent which can donate an amine group, for example urea, in order to form a phosphoamine linked to the polycarboxylic acid. An exemplary substituted polycarboxylic acid is citric phosphoamine.

In one embodiment of the present invention, the yeast genus Candida can be used in the method of preparing PPPBs presented herein. In a related embodiment Candida utilis is used in the method of the present invention. One exemplary strain of C. utilis has been deposited with the American Type Culture Collection (ATCC), 12301 Parklawn Drive, Rockville, Md. 20852, USA, and assigned registration No. 9950.

Treatment of yeast strains starting with low concentrations of stressor and then with ever increasing concentrations of the same stressor induces an adaptive response in surviving yeast strains that protects these yeast from the lethal effects of a subsequent challenge with higher concentrations of the same stressor (Jamieson et al. (1996) FEMS Microbiol Lett 138:83-88). It has been suggested that the basis for such adaptive responses rests in increased expression of genes that encode protective enzymes and repair enzymes (Davies et al. (1995) Arch. Biochem. Biophys. 317:1-6). The stress responses of yeast is reviewed in Mager and Ferreira (1993) Biochem. J. 290:1-13. Standard procedures known to one skilled in the art can be used to grow and stress yeast in a process such as the following.

In one demonstration, a sterilized agar slant is inoculated with an actively growing culture of Candida sp. and incubated until the yeast cell density is adequate to be used as an inoculum. The yeast cell strain is grown in culture medium according to methods known to one skilled in the art. Typical growth media comprises, for example, a yeast cell extract, peptone, and glucose (YPG). The pH of the culture medium is maintained from between 6.0 to 8.0, for example, at pH 6.5. The cultivation temperature is maintained between 28° C. to 35° C., for example at 30° C. Cultivation requires aeration of the inoculum. The vessel or flask housing the inoculum may be agitated on a rotary shaker at about 250 rpm or by stirring the inoculum with a stirring apparatus located within the vessel.

Standard growth curves are prepared according to methods well known in the field. Briefly, liquid culture medium is inoculated with the yeast cell culture and incubated. At regular time intervals, samples of the yeast cell culture are obtained and analyzed for growth, using methods well known in the field, including cell counters and absorbance measurements.

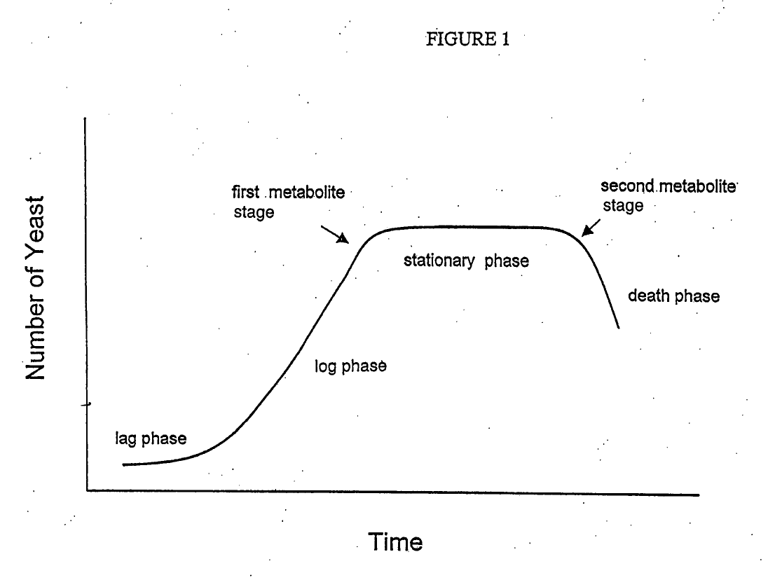

As shown in FIG. 1, yeast cells of the genus Candida sp. typically show a characteristic growth pattern when inoculated into a fresh culture medium. There is an initial lag phase, and then growth commences in an exponential fashion (log phase). As essential nutrients are depleted or toxic fermentation products build up, growth ceases and the yeast cell population enters the stationary phase. The point at which the yeast cells enter the stationary phase is called the first metabolite stage. If incubation continues, yeast cells may begin to die and the population is said to be in the death phase. The point at which the yeast cells enter the death phase is called the second metabolite stage.

The pH of the liquid yeast cell culture medium is maintained at 6.5 and measured twice per day throughout the fermentation process using an automated pH meter contained within the fermentation vat. The pH of the medium has a tendency to become acidic until the first metabolic stage is reached, at approximately 7 days, and concentrated NaOH must be added to the yeast cell culture in order to maintain a pH of 6.5. After the first metabolite stage is reached the pH of the medium has a tendency to become basic. An acidic compound, for example citric phosphoamine, must be added in order to decrease the pH of the medium to about a pH of 6.5. Once the pH of the fermentation medium reaches pH 8.0, at about 10 days post inoculation, the fermentation process is terminated.

Yeast cells at the first metabolite stage of growth, are subjected to a number of stressors which are added to the yeast cell culture. These stressor compounds induce the production of cellular stress responses within the yeast cells which cause the surviving cells to become resistant to the stressors. Upon exposure to a stressor, several physiological events occur in yeast cells that allow them to adapt and become resistant to the particular stressor to which they are exposed. The overall result of these events is that the yeast cells rapidly begin synthesizing detoxification (stress response) proteins while synthesis of other peptides is suppressed. The type of stressor, and the duration and intensity of stress can affect the quantity and quality of the synthesis of a particular detoxification protein.

A further characteristic of stress-tolerant yeast phenotype is “translational-tolerance,” which relates to both the rate of protein synthesis in general, the extent of protein synthesis, or both, by a yeast cell after exposure to a stressor. In normal cells (those not yet made stress-tolerant), protein synthesis rates drop upon exposure to a stressor and require considerable time to return to normal. In stress-tolerant phenotypes, the recovery of protein synthesis is considerably faster.

The present invention makes use of the ability of yeast cell cultures to adjust to and recover from the addition of stressor molecules. Stressors of the present invention include, but are not limited to, terpenes, natural plant resins, carbohydrates, lipids, natural oils, animal or plant DNAs, allergens, synthetic or naturally occurring toxins, heavy metals, inorganic chemical compounds, organic chemical compounds and any other molecule, composition, compound or substance that may induce a stress response in yeast cells. Stressors from any or all of these groups may be added. In one embodiment, one stressor from each group is added sequentially.

In one example, the first stressor is a terpene. Any terpene may be used, including geraniol, citral, pinene, bomeol, citronellol and γ-terpinene. In an exemplary embodiment, camphor is used. In one example, the second stressor is a natural pine tree resin. Any pine tree resin may be used. In an exemplary embodiment, colofonic, the resin of the tree Pinus palustris, which is high in abietic acid, is used. In one example, the third stressor is a source of starch. Any source of starch may be used, including that isolated from potatoes, beans and rice. In an exemplary embodiment, milled soy beans are used. The fourth stressor may be a source of natural oils. Any source of natural oil may be used, including corn, linseed, palm, olive, canola, soybean, vemonia, and castor. In an exemplary embodiment, milled castor beans are used. The fifth stressor is animal DNA. Any animal DNA may be used, including DNA from birds and mammals. In an exemplary embodiment, DNA extracted from chicken fertilized egg yolk is used. The stressors are added to the yeast sequentially. The stressors may be added in any order. For example, in one embodiment, the stressors are added in an order of increasing complexity, such as molecular complexity.

The following stressing procedure is used for each stressor. Stressing yeast cells involves two steps: an inhibition step and a selection step.

1. Inhibition: The stressor is added to the yeast cells at the first metabolite stage of growth in increasing amounts until yeast growth is inhibited by 50 to 90 percent. Inhibition of yeast cell growth is determined by methods familiar to someone skilled in the art.

2. Selection: In order to select for modified yeast cells that are capable of growing in the presence of high concentrations of the applied stressor, a sample of the inhibited yeast cell culture is inoculated in medium containing this inhibiting concentration of stressor. This culture is then cultivated for one week to select for the live (successful) strains of yeast cells. This selection process is repeated many times at the same concentration of stressor until the sample reaches the first metabolite stage of growth in the one week growing period. One skilled in the art would know how many times this selection process needs to be repeated, for example, from 15 to 25 times.

Samples of the final yeast cell culture are preserved for future use by storage methods familiar to someone skilled in the art, such as dehydration or lyophilization.

The inhibition step (1) and selection step (2) are repeated using the same stressor until a maximum concentration of a particular stressor is obtained. Once the yeast cells no longer grow to the first metabolite stage, the last yeast cell culture to be successfully grown to the first metabolite stage is used as the starting point for either the next stressor addition or for fermentation. The concentration of stressor used for this culture is maintained in the next step of the procedure.

Once the final stressor concentration has been determined, the final modified yeast cell culture is used in the final fermentation process. This resultant strain of yeast cell can grow actively and survives well even under the predetermined high concentrations of stressors used. The fermentation process is carried out in an appropriately-sized, sealed fermentation vessel. In one example, the vessel has a 10 L capacity. About 0.1 to 20%, for example, about 15% by volume, of inoculum is added to the production medium. The remainder of the volume comprises the fermentation medium. Any technique known by someone skilled in the art for introducing the inoculum in an active metabolic state and does not cause contamination of the culture is acceptable for use with this procedure.

To control foaming, it may be desirable to add an anti-foaming agent to the medium, such as a silicone de-foamer, at a concentration of 0.01 to 1% by volume. The production medium will be the same medium as used during the stressing stage, supplemented with the stressors at their final concentrations as determined in the previous step.

The fermentation medium is brought to a temperature of about 28° C. to 30° C., and can be approximately 30° C. Fermentation is carried out until the second metabolite stage of growth. The length of time required to reach this stage depends upon the PPPB compositions of the fermentation medium, temperature, quantity of cells in the inoculum, and concentration of stressors used. Typically, the fermentation process is conducted for approximately 8 to 10 days. It is desirable to maintain the pH in the range of 6.0 to 8.0, for example, 6.5. During the initial period of fermentation, the pH can slowly decrease into the acid range; wherein it can be adjusted with a base, such as NaOH. As fermentation proceeds, the pH can begin to increase wherein it can then be adjusted back to the appropriate range using a polycarboxylic acid, such as citric phosphoamine.

Once the second metabolite stage of growth is obtained, fermentation is stopped and a polycarboxylic acid is added to provide the backbone for the biopolymers. In one embodiment, citric phosphoamine is used. The PPPBs generated as a result of this process have the structure depicted in Formula I. It should be understood that other polycarboxylic acids or derivatives thereof can be used, for example, citric acid, tartaric acid or tartaric phosphoamine. The use of these polycarboxylic acids alone or in combination should not limit or restrict the scope of this invention in any way. The resulting mixture can be referred to as a production culture.

The present invention also provides for the preparation of pPPPBs, which can be used as immunomodulators and/or biological response modifiers. These pPPPBs are prepared by phosphorylation of the PPPBs. A phosphorylating agent is added to generate the pPPPBs. Any phosphorylating agent may be used, including phosphoric acid and ATP. In one embodiment, the phosphorylating agent is phosphoric acid. A source of amino groups is also added. Various possible amino sources may be added, as understood by one skilled in the art, including urea and ammonia. In one embodiment, the amino source is urea.

Following fermentation, the yeast cell walls are ruptured using techniques known to a person skilled in the art, including ultrasound, compression, and freezing. The following procedure is one example of a method for rupturing yeast cell walls. Following fermentation, the pH is adjusted to 7.0 and phosphoric acid, urea and pepsin are added to the yeast cells and culture medium. The mixture is then left to sit at room temperature for 24 hours in a sterile container and then frozen at −20° C. for 1 week. The mixture is returned to room temperature, mixed and allowed to settle for 1 to 2 days. The clear portion of the mixture is poured off and retained. The remainder of the liquid is filtered to remove any particulate matter. The two clear solutions are combined, and any remaining microbial debris is removed from the solution by filtration using, for example, a filter membrane or filter paper with a 0.22 μm mesh size. Numerous methods of filtration are familiar to a worker skilled in the art and may be used in the method of the present invention.

Acetone, or a similar solvent, is added to the clear sterile filtrate in a ratio of aproximately 2: 1, the acetone:filtrate solution is mixed and frozen at approximately -20° C. for 1 week. The clear solution is poured off and the solid precipitate is retained. A mixture of calcium phosphate dibasic and calcium sulfate in a 2:1 ratio is added to the solid precipitate, 10 g/g of frozen solid, and mixed at room temperature. The resulting complex solid is passed through a #20 mesh filter to obtain uniform particles and then air dried in an oven set at a temperature of no more than 50° C. The dried solid contains pPPPBs.

Characterization of Polysubstituted Polycarboxylic Phosphoamide Biopolymers

Once prepared the pPPPBs of the present invention can be analyzed using standard in vitro and in vivo techniques known to workers skilled in the art in order to demonstrate the physical and biological activity characteristics of these biopolymers.

In Vitro Assays

The protein and carbohydrate content of the isolated pPPPBs can be determined using standard protein and carbohydrate assays. The protein content of the pPPPBs of the present invention is greater than 0.1% by weight. Similarly the carbohydrate content of the pPPPBs of the present invention is greater than 0.1% by weight. The pPPPBs can be further characterized using standard techniques including electrophoretic (eg SDS-PAGE) and chromatographic (e.g. HPLC) analysis.

The biological activity of the pPPPBs of the present invention can be demonstrated using various methods including, but not limited to, the rosette inhibition test (Morton et al. 1974 Nature 249(456):459-460). This assay is used to demonstrate the ability of the pPPPBs to activate T-lymphocytes. Active pPPPBs demonstrate a significant increase in the number of activated T-lymphocytes forming rosette patterns in comparison to the number activated in the absence of pPPPBs. This test is described in greater detail in Example III provided herein.

In Vivo Assays

Standard toxicity tests can be used to demonstrate that pPPPBs are non-toxic in mammals. For example, LD₅₀ assays using mice demonstrated that doses as high as 15 mg/g body weight per day did not produce any toxic effects. This dose is 100 times higher than the recommended estimated dosage for humans.

In analyzing the immune and inflammatory response of animals treated by pPPPBs various criteria are measured using techniques well known to workers skilled in the art, including, but not limited to: monocyte mobilization; polymorphonuclear cell activity; concentration of cytokines including IL-1 and INF-γ; concentration of colony stimulating factors including GM-CSF, G-CSF and M-CSF; hematopoeitic activity; and phagocytic activity (phagocytic index). In each case the pPPPBs are shown to improve healing and/or decrease recovery time by modulating inflammatory and/or immune responses in test animals in comparison to untreated animals.

Clinical Trials

Following the preliminary testing in vitro and in animals, the pPPPBs are tested in clinical trials using consenting patients that suffer from various diseases, infections and/or traumas. Examples of such clinical trials are described in greater detail in Examples VI to XI provided herein.

In demonstrating the biological activity of the pPPPBs in patients various criteria are measured using techniques well known to workers skilled in the art, including, but not limited to: determination of frequency of infection; determination of duration of hospital stay; calculation of phagocytic index; hematic analysis including percent hemoglobin (Hb.%), hematocrit (Hto%), erythrocyte count, platelet count, leukocyte count, neutrophil count, lympocyte count, monocyte count, eosinophil count and basophil count; liver and renal function tests. The results of these tests demonstrate that the pPPPBs of the present invention can improve healing and/or recovery time in patients by modulating inflammatory and/or immune responses in patients in comparison to untreated patients.

Use of Polysubstituted Polycarboxylic Phosphoamide Biopolymers

The pPPPBs of the present invention can be used as therapeutic and/or prophylactic agents, either alone or as adjuvants, to modulate the immune system in humans and animals in order to allow the patient's immune system to work more efficiently to combat diseases and/or infections and to promote healing, such as for bums, wounds, concussions and surgical incisions due to surgery to remove tumors and open trauma wounds. In one embodiment the pPPPBs act to enhance the immune response of the patient. In a related embodiment the pPPPBs of the present invention can be used as therapeutic and/or prophylactic agents, either alone or as adjuvants, to modulate the inflammatory system in humans and animals and thereby promote healing and combat diseases and/or infection.

The use of the term “wounds' refers to wounds that are internal or external bodily injuries or lesions caused by physical means, such as mechanical, chemical, bacterial, viral, or thermal means, which disrupt the normal continuity of structures. Such bodily injuries include contusions, wounds in which the skin is unbroken, burns, incisions, wounds in which the skin is broken by a cutting instrument, and lacerations, wounds in which the skin is broken by a dull or blunt instrument. Wounds may be caused by accidents or by surgical procedures.

The pPPPBs are particularly useful for treatment of trauma patients. Treatment with the pPPPBs of the present invention have been shown to be particularly effective in mobilizing a patient's normal immune defenses.

The pPPPBs of the present invention may be used to treat patients with immunosuppression caused by age, malnourishment, diseases, such as cancer or AIDS, or medical treatments, such as chemotherapy or radiotherapy. For example, the pPPPBs can be used to pre-initiate the metabolic immune response in patients who are undergoing chemotherapy or radiation therapy, or who are at a heightened risk for developing secondary infections or post-operative complications because of a disease, disorder, or treatment resulting in a reduced ability to mobilize the body's normal metabolic responses to infection.

The pPPPBs can also be used for the prevention and treatment of infections caused by a broad spectrum of micorbial pathogens, including bacterial, fungal, viral and protozoan pathogens. As an example, the prophylactic administration of the pPPPBs in this invention to a person undergoing surgery, either pre-operatively, intra-operatively and/or post-operatively, will reduce the incidence and severity of post-operative infections in both normal and high-risk patients. In patients undergoing surgical procedures that are classified as contaminated or potentially contaminated (e.g., gastrointestinal surgery, hysterectomy, cesarean section, transurethral prostatectomy) and in patients in whom infection at the operative site would present a serious risk (e.g., prosthetic arthroplasty, cardiovascular surgery), concurrent initial therapy with an appropriate antibacterial agent and the present pPPPBs contained in this invention will reduce the incidence and severity of infectious complications. The pPPPBs of the present invention can be used to promote wound healing such as burns and other physical injury.

A therapeutically effective dose of the pPPPBs of the present invention may be administered to the host in a manner familiar to someone skilled in the art. In one embodiment, administration of the pPPPBs is oral. Other routes of administration for the pPPPBs may be parenterally (including intravenously, intramuscularly, and subcutaneously), intracisternally, intravaginally, rectally, intraperitoneally, locally (including the use of powders, ointments, or drops), nasally (including sprays or drops), topically, or entericly.

One embodiment of the present invention, therefore, is a method of treating a patient comprising the step of administering a therapeutically affective does of pPPPBs. A worker skilled in the art would readily appreciate that this treatment can involve administration of a single dose of pPPPBs or multiple doses.

Pharmaceutical Compositions and Formulations

The pPPPBs of the present invention may be formulated in any manner that makes them suitable for administration. Formulations may take the form of solid, semi-solid, lyophilized powder, or liquid dosage forms, such as, for example, tablets, pills, capsules, cachets, lozenges, powders, sustained-release formulations, solutions, dispersions, suspensions, emulsions, suppositories, retention enemas, creams, ointments, lotions, aerosols, or the like.

The pPPPBs may be formulated with pharmaceutically acceptable excipients, carriers, adjuvants, solvents, or vehicles in accordance with conventional pharmaceutical practice.

In solid formulations, the pPPPBs of the present invention can be admixed with one or more diluents, excipients such as a saccharide or cellulose preparation, fillers or extenders, humectants, flavoring agents, solubilizers, lubricants, suspending agents, binders such as starch paste or methyl cellulose, preservatives, disintegrating agents, solution retarders, wetting agents, adsorbents, buffering agents, or encapsulating materials, coatings, or shells. In powders, the carrier is a finely divided solid which is in a mixture with the finely divided active component. In tablets, the active component is mixed with the carrier having the necessary binding properties in suitable proportions and compacted in the shape and size desired.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Included in the formulation procedures of the compositions in this invention are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Liquid form preparations include solutions, suspensions, emulsions, syrups, and elixirs, being supplied either in liquid form or in a dried form suitable for hydration.

A solution would ideally have a concentration of from about 1 mg/ml to about 100 mg/ml. In liquid formulations, the pPPPBs of the present invention may contain inert diluents commonly used in the art such as water, aqueous saline, aqueous dextrose, glycerol, ethanol, or other solvents. Besides such inert diluents, the pPPPBs can also include adjuvants, such as wetting agents, emulsifying and suspending agents, solubilizing agents, and sweetening, flavoring, or perfuming agents. Suspensions, in addition to the active compounds, may contain suspending agents.

The pPPPBs of this invention can also be administered in the form of liposomes. As is known in the art, liposomes are generally derived from phospholipids or other lipid substances. Liposomes are formed by mono or multi-lamellar hydrated liquid crystals that are dispersed in an aqueous medium. Any non-toxic, physiologically acceptable, and metabolizable lipid capable of forming liposomes can be used. The present pPPPBs in liposome form can contain stabilizers, preservatives, excipients, and the like. Examples of lipids that may be used are phospholipids and phosphatidyl cholines (lecithins), both natural and synthetic. Methods of forming liposomes are well known in the art.

The pPPPBs may also be administered by inhalation, in the form of aerosol particles, either solid or liquid. Such particles may be of respirable size and sufficiently small to pass through the mouth and larynx upon inhalation and into the bronchi and alveoli of the lungs. In general, particles ranging from about 1 to 10 microns (>m) in size are respirable.

Formulations containing respirable dry particles of micronized active agent may be prepared by grinding dry active agent and passing the micronized pPPPBs through a 400 μm mesh screen to break up or separate out large agglomerates. The solid particulate form of the active agent may contain a dispersant to facilitate the formation of an aerosol. A suitable dispersant is lactose, which may be blended with the active agent in any suitable ratio (e.g., a 1:1 ratio by weight).

Any solid particulate medicament aerosol generator may be used to administer the solid particles. Such generators, such as the DeVilbiss™ nebulizer (DeVilbiss Co., Somerset, Pa.), produce particles that are respirable, and generate a volume of aerosol containing a predetermined metered dose of a medicament at a rate suitable for human administration. Liquid pPPPBs for inhalation comprise the active agent dispersed in an aqueous carrier, such as sterile pyrogen free saline solution or sterile pyrogen free water. If desired, the pPPPBs may be mixed with a propellant to assist in spraying the pPPPBs and forming an aerosol.

The present pPPPBs is generally administered to an animal or a human in an amount sufficient to produce immune system enhancement. For humans, a daily dose range of about 10-50 mg/kg may be used, however higher doses are well tolerated. The amount necessary to induce immune system enhancement will vary on an individual basis and be based at least in part on consideration of the individual's size, the severity of the symptoms, and the results sought. Determination of the proper dosage for a particular situation is within the skill of the art, for example, see Remington's Pharmaceutical Sciences (1980). For convenience, the total daily dosage may be divided and administered in portions during the day if desired.

The formulation can, if desired, also contain other compatible therapeutic agents.

Kits

The present invention additionally provides for therapeutic kits containing one or more pharmaceutical composition as described herein. The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an infected area of the animal, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

Various embodiments of the present invention are described in further detail in the following non-limiting examples. It is to be understood that the examples described below are not meant to limit the scope of the present invention. It is expected that numerous variants will be obvious to the person skilled in the art to which the present invention pertains, without any departure from the spirit of the present invention. The appended claims, properly construed, form the only limitation upon the scope of the present invention.

### EXAMPLES

### EXAMPLE I

### Preparation of pPPPBs

The preparation of exemplary pPPPBs is demonstrated herein. A tube containing solid YPG (1.5 g/L yeast cell extract, 5 g/L peptone, 10 g/L glucose) medium is inoculated with C. utilis ATCC -9950 and incubated at 28° C. to prepare a yeast cell stock. The purity of the stock is verified after 24 and 48 hours by macroscopic and microscopic observation.

A standard growth curve is prepared. Flasks (250 ml) containing 100 ml liquid YPG medium are inoculated with a loopful of the yeast cell stock and placed on a rotary shaker at 250 rpm in an incubator set at a temperature of 28° C. At 24 h and continuing until 60 h after inoculation, 2 ml samples are taken every 8 h and analyzed for growth using a method known to a person of skill in the art, for example by monitoring the pH. FIG. 1 shows a standard growth curve for yeast cells, Candida sp. The abscissa represents days of fermentation, the ordinate log growth of yeast cells.

Once the yeast cells are at the first metabolite stage of growth, they are sequentially subjected to five different stressors according to the following procedure. A flask (250 ml) containing 100 ml of liquid YPG medium is inoculated with a loopful of the yeast cell stock and placed on a rotary shaker at 250 rpm at 28° C. Once the yeast cells reach the first metabolite stage of growth, camphor is added, beginning with 10 mg and increasing the amount until yeast cell growth is inhibited by at least 90 percent as compared to growth in the absence of stressor. Inhibition of growth rate is determined by measuring turbidity or pH. At this point, the concentration of camphor in the YPG is kept constant, and new medium is then prepared at this concentration of camphor (called YPGC medium).

A tube containing 5 ml YPGC medium is then inoculated with a loopful of the inhibited yeast cell culture and placed on a rotary shaker at 250 rpm at 28° C. for one week. Growth is analyzed by measuring turbidity or pH. A loopful of this yeast cell culture is then used to inoculate a second tube containing 5 ml YPGC medium and placed on a rotary shaker at 250 rpm at 28° C. for one week. Again, growth is analyzed using methods known to a person skilled in the art, for example turbidity or pH. A loopful of this yeast cell culture is then used to inoculate a third tube containing 5 ml YPGC medium and placed on a rotary shaker at 250 rpm at 28° C. for one week.

This procedure of successive inoculations is repeated 15 to 20 times, until growth measurements of the yeast cell cultures indicate that the growth of the cell cultures has reached the first metabolite stage. At this point, 100 ml of fresh YPGC is inoculated with the final yeast cell culture. Once the yeast cells are again at the first metabolite stage of growth, samples are lyophilized for future use, as described previously. With the remainder of the yeast cells, the concentration of the first stressor, camphor, is increased until yeast cell growth is inhibited by at least 90 percent. At this point, the concentration of camphor in the YPGC is kept constant, and 15 to 20 successive inoculations are made again until growth measurements of yeast cell cultures indicate that growth is again at the first metabolite stage.

This procedure of increasing camphor concentrations until yeast cell growth is inhibited by 90 percent, then making successive inoculations at a constant concentration of camphor until the yeast cells reach the first metabolite stage, is repeated until the yeast cells fail to return to the first metabolite stage. At this point, the last lyophilized yeast sample that was able to reach the first metabolite stage of growth is reconstituted in 100 ml YPGC at the corresponding concentration of camphor, in this example, 9 g/L.

This procedure is then repeated with a second stressor, Colofony (Hercules, Mexico City), a resin from pine trees (Pinus palustris) rich in abietic acid. This resin is dissolved in ethyl alcohol and added to the yeast cells growing in YPGC medium in increasing amounts beginning at 100 mg/L. Following the above procedure, a final concentration of 9 g/L of YPGC (called YPGCR medium) is reached.

The procedure was then repeated with a third stressor, milled soy beans. The soy beans were milled in water to a smooth consistency using a blender. The milled soy beans were added to the yeast cells growing in YPGCR medium in increasing amounts beginning at 100 mg/L and following the above procedure to reach a final concentration of 10 g/L of YPGCR (called YPGCRS medium).

The procedure is repeated with a fourth stressor, castor beans (Ricinus communis). The castor beans are milled in water to a smooth consistency using a blender. The milled castor beans are added to the yeast cells growing in YPGCRS medium in increasing amounts beginning at 100 mg/L and following the above procedure to reach a final concentration of 10 g/L of YPGCRS (called YPGCRSC medium).

The procedure is repeated with a fifth and final stressor, DNA from fertilized chicken egg yolks. The egg yolks are frozen for one week, returned to 20° C., mixed with saline solution (8.5 g/L), and filtered. The DNA is obtained by extraction using methods known to those skilled in the art. The DNA is suspended in water and added to the yeast cells growing in YPGCRSC medium in increasing amounts beginning at 100 mg/L and following the above procedure to reach a final concentration of 3 g/L of YPGCRSC (called YPGCRSCD medium).

The final fermentation step requires using a loopful of the final yeast cell culture growing in YPGCRSCD medium to inoculate 100 ml of fresh YPGCRSCD medium in a 250 ml flask. This is repeated to obtain four flasks, which are placed on a rotary shaker at 250 rpm at 28° C. and grown to the first metabolite stage, as determined by measuring growth of cell cultures as described previously. The 100 ml of growing yeast cell culture is added to 10 L of YPGCRSCD medium in a 14 L fermenting apparatus and maintained at 30° C. Growth of the culture is monitored by pH and by using an oxygen electrode to measure oxygen in the medium. The volume of the fermentation batch, containing YPGCRSCD medium and yeast cells, is maintained at 10 L using sterile saline solution.

The pH of the fermentation mixture is maintained at pH 6.5. At the beginning of the fermentation (approximately 5 days), sodium hydroxide is added, for example at an amount of about 5 M; towards the end of the fermentation, a polycarboxylic acid is added, for example citric phosphoamine, at an amount of about 7 M, in order to maintain the proper pH of about 6.5 during fermentation.

Once the oxygen level drops (at about 8 to 10 days), the fermentation is stopped by the addition of 7 M citric phosphoamine, until the fermentation mixture reaches a final pH of 4.0. At this time, the fermenting apparatus is opened and discharged. About 200 ml of a 75 to 80 percent solution of phosphoric acid is then added, along with 35 g of pepsin and 50 g of urea. The mixture is allowed to sit at room temperature for 24 h and is gently stirred.

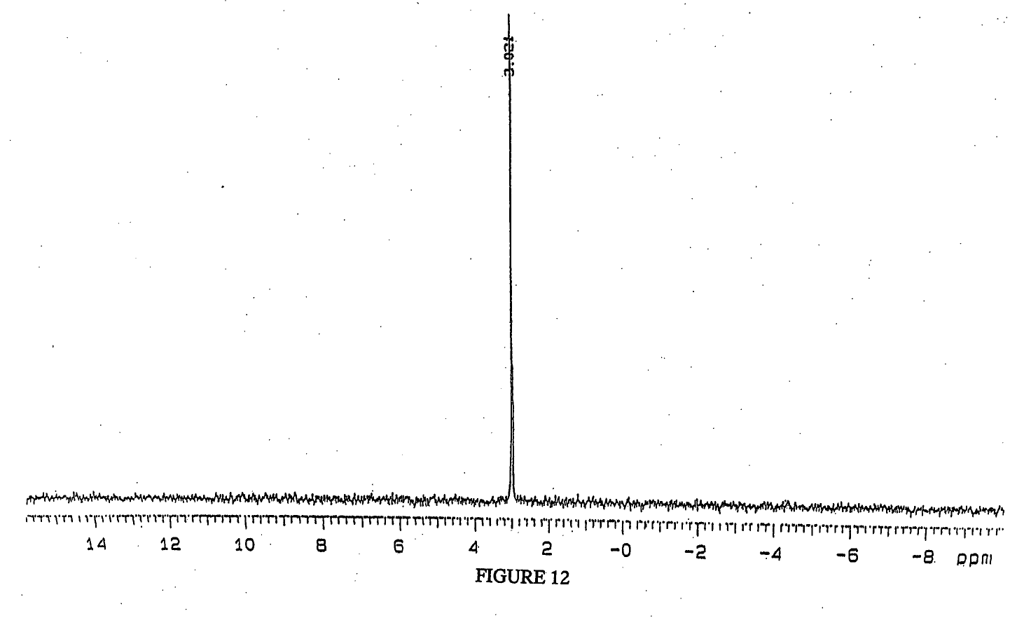

Following this procedure the yeast cell walls are ruptured by incubating the mixture at −20° C. for 7 days, until all cells are frozen. The mixture is returned to 20° C. and the clear portion is separated and placed in a separate flask. The remainder of the thawed mixture is passed through cheese cloth to remove large particulate matter. The filtrate from this procedure is added to the clear solution, mixed and passed through a 0.22 μm filter to remove any further cell debris. The fermentation product that resulted from this phosphorylation procedure is approximately 3 percent phosphorylated, as measured by ³¹P-NMR (FIG. 12 and the phosphovanadate method (Table 13). The solution was then adjusted to a final pH 7.0 using 5 M sodium hydroxide.

The phosphorylated product is treated with 2 volumes of acetone to form a gummy layer. This mixture is placed at −20° C. for 7 days until the bottom layer is frozen solid and the upper aqueous layer is then drained off. A mixture of calcium phosphate dibasic and calcium sulfate, in a 2:1 ratio, is added to the bottom layer, 10 g per gram of frozen solid, and mixed at room temperature to form a complex solid. The resulting solid is then passed through a #20 mesh filter in order to obtain uniform particles. The particles are dried at 45° C. with air ventilation for 24 h.

### EXAMPLE II

### Characterization of pPPPBs

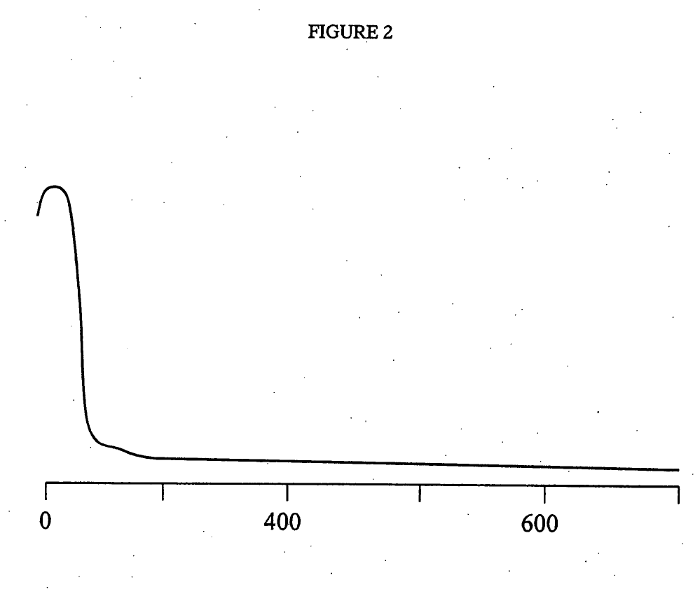

Representative analytical data profiles of the pPPPBs are shown in FIGS. 2 to 5, 12 and 13.

UV Absorption Spectrum for pPPPBs

The UV spectrum of the pPPPBs can be obtained using the following method. Approximately 5 g of the pPPPBs-calcium phosphate/calcium sulphate mixture is weighed and 10 ml of phosphate buffered saline, pH 7.0, added. The solution can be mixed with a vortex and allowed to settle for 10 minutes. The clear solution is poured off and 20 ml of acetone can be added to the precipitate, mixed and the solution refrigerated over night. The solution can then be filtered using a porous glass filter, the precipitate air dried and dissolved in 5 ml of water. The UV spectrum of this sample can be measured in a standard spectrophotometer.

The UV spectrum may also be measured for the pPPPBs in the absence of the calcium phosphate/calcium sulfate matrix in a similar manner. However, in this procedure 500 mg of starting material can be added to 5 ml of phosphate buffered saline. The clear solution can be poured off and 10 ml of acetone added to the precipitate and refrigerated, filtered, dried and dissolved in 5 ml of water as above.

A UV absorption spectrum of the pPPPBs is shown in FIG. 2. It is possible to observe a band centered at 196 nm which is the maximum absorbance peak for the pPPPBs produced using the method of the outlined herein.

HPLC Analysis for the Amino Acid Content of pPPPBs

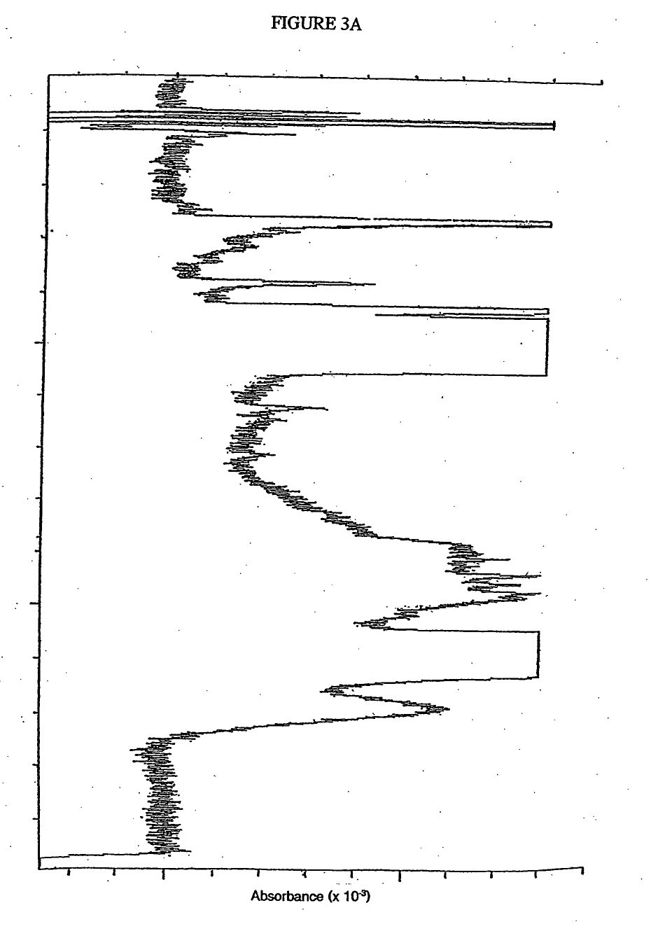

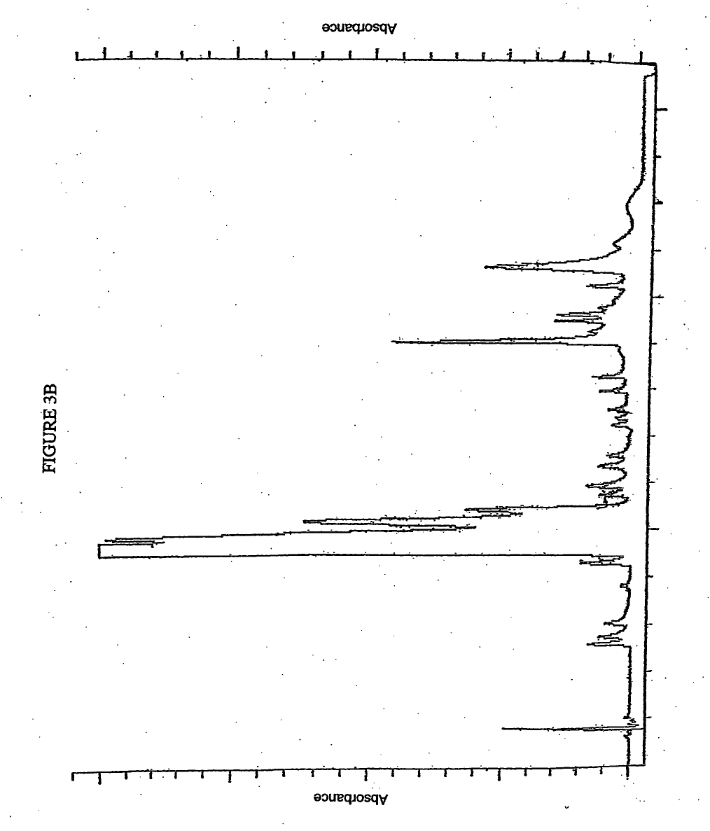

Amino acid analysis of the pPPPBs described can be determined using the following procedure. Lyophilized yeast cell extract samples obtained from the final fermentation procedure can be hydrolyzed in 6 N H₂SO₄ in evacuated sealed Pyrex™ tubes at 110° C. for 24 hours. The hydrolyzed products can then be derivatized with 4-dimethylaminoazobenzene-4′-sulfonyl chloride (dabsyl chloride) using the method described in Gorbics et al., 1994 (J. Chromatogr. 676(1):169-176). Derivatized products of the pPPPBs can be examined on a Beckman system Gold HPLC apparatus, using an Ultrasphere-dabsyl C₁₀ column (250×4.6 mm). A similar procedure can used to hydrolyze and derivatize a known amount of the standard protein, albumin. A trace showing the protein content of pPPPBs, is shown in FIG. 3(A) and that for albumin, in FIG. 3(B). The retention times for the amino acids serine, threonine, arginine, proline, valine, lysine, histidine, and tyrosine shown in FIG. 3(A) are, 9.65, 10.33, 10.89, 12.54, 13.00, 18.12, 18.94, 19.62 and 20.42 minutes, respectively. A worker skilled in the art will recognize that other amino acids may be contained in the pPPPBs. These retention times may be determined from FIG. 3(B), the trace for albumin, whose amino acid sequence is known.

SDS-PAGE Analysis to Determine the Protein Molecular Weight of pPPPBs

Sodium dodecyl sulfate polyacrylamide gel electrophoresis (SDS-PAGE) can be performed using a Phast System™ (Amersham Pharmacia Biotech). Samples of the pPPPBs can be loaded onto and electrophoresed in a manner familiar to someone skilled in the art of homogeneous 20% polyacrylamide gels (0.45×43×30 mm) using SDS buffer strips. SDS gels can be developed according to the methods detailed in Butcher and Tomkins, 1985 (Anal. Biochem. 148(2):384-388). Phosphorylase B (97 kDa), bovine serum albumin (66 kDa), ovalbumin (45 kDa), carbonic anhydrase (30 kDa), trypsin inhibitor (20.1 kDa) and α-lactalbumin (14.4 kDa) can be used as standard molecular weight markers. Band positions of standards and of the protein bands of the pPPPBs can be determined by means of a LKB Ultrascan Laser Densitometer. Two distinct bands on the SDS-gel represent the protein profile for the pPPPBs. Molecular weights ranged from 14 to 16.3 kDa and 16.7 to 18.0 for the lightest and heaviest protein bands, respectively as determined from 3 separate preparations of the purified (Table 13).

¹H-NMR, ¹³C-NMR and ³¹P-NMR Analysis of pPPPBs

NMR can be performed using Varian Unit Plus™ equipment and standard NMR methodology familiar to a person skilled in the art. The reference standards D₂O, dioxane and K₂HPO₄ can be used and the intensities for each are 500 KHz, 125 KHz and 121 kHz for ¹H-NMR, ¹³C-NMR and ³¹P-NMR, respectively.

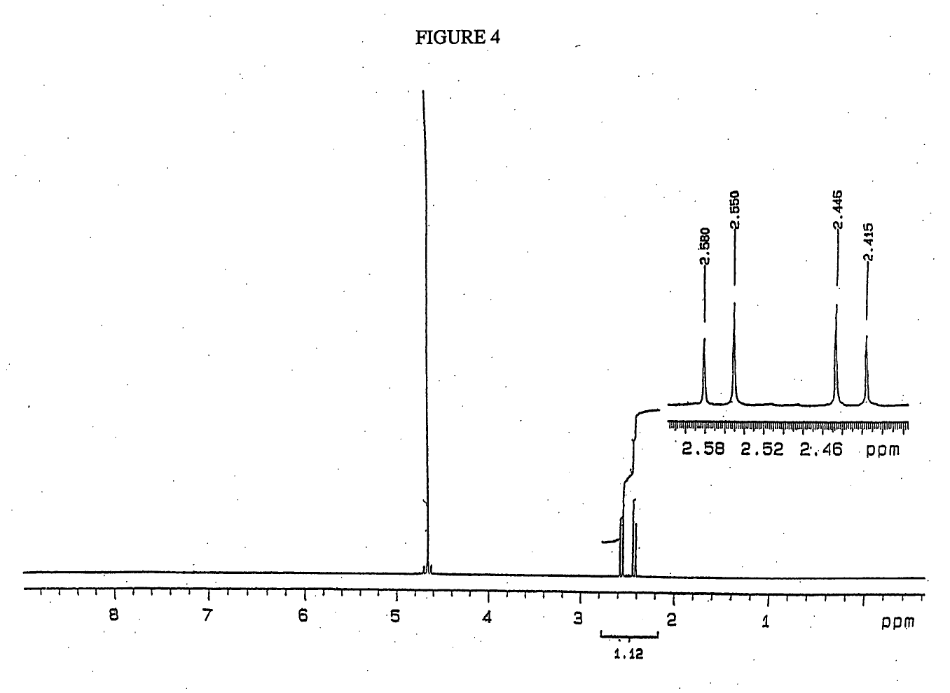

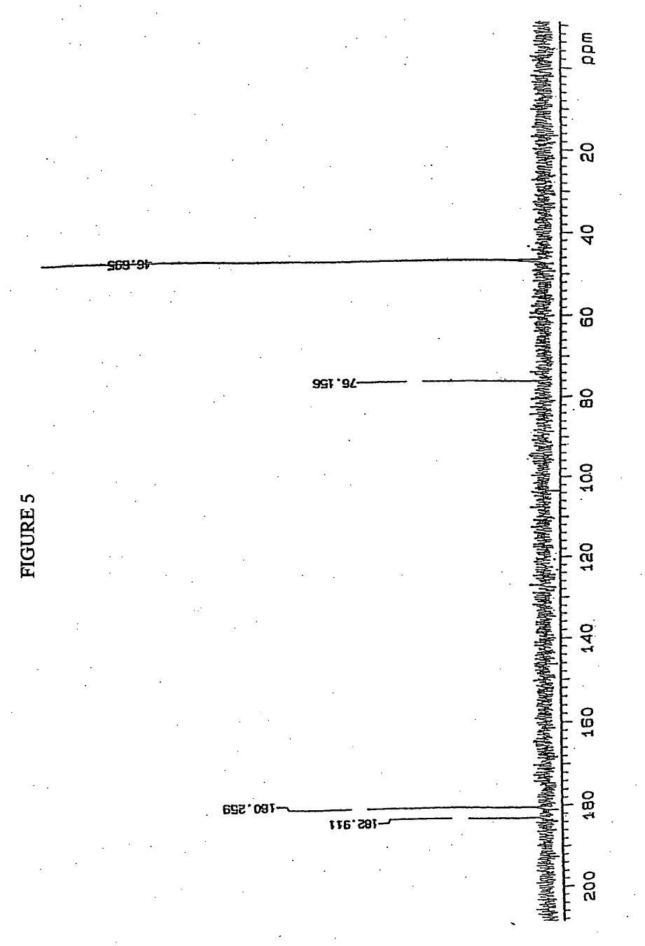

A trace showing an ¹H-NMR analysis of pPPPBs, is shown in FIG. 4. A peak at 3.5 ppm indicates the presence of carboxyl groups. Peaks at 2.580, 2.550, 2.446 and 2.415 indicate the presence of methylene groups. A trace showing the ¹³C-NMR analysis of the pPPPBs is shown in FIG. 5. A peak at 76.156 ppm indicates the presence of a phosphoamide and a peak at 46.695 ppm indicates the presence of carboxyl groups. A trace showing the ³¹P-NMR analysis of the pPPPBs is shown in FIG. 12. A peak at 3.021 indicates the presence of a phosphate group.

Protein Content of pPPPBs

Protein content of the isolated and purified pPPPBs (at least n=3 separate isolations) are shown in Table 13. The total protein content of isolated and purified pPPPBs can be determined by the Lowry method as modified by Smith et al. 1985. A Bicinchoninic Acid Kit (BCA-1) can be used as an alternative to the Folin-Ciocalteu reagent to follow the reduction of Cu(II) to Cu (I) in a concentration dependent manner in the presence of protein. Absorbance can be measured at 562 nm using a spectrophotometer and protein determined with reference to a standard curve using BSA protein. Sample solutions containing the pPPPBs can be prepared by adding 5 g of either isolated or purified pPPPBs to 25 ml of 0.05 N HCl. This solution is mixed and incubated for 30 min at 37° C. Aliquots are then assayed for protein content. Protein content for isolated and purified pPPPBs ranges from 0.1-0.5% and 2-2.8%, respectively (Table 13).

Isoelectric Focusing Analysis to Determine the Isoelectric Point (pI) of pPPPBs

Isoelectric focusing analysis can be used in order to determine the isoelectric point (pI) of the pPPPBs. Phast Gel IEF 3-9 slabs (0.35×43×50 mm, pH range 3-9) are run in a Phast System™. Formation of the pH gradient and focusing of the sample of the pPPPBs takes approximately 45 min under a constant power of 3.5 W and IEF gels are then developed. Isoelectric points of the focused samples of purified (n=3) are shown in Table 13. The pPPPBs once focused have a final isoelectric point that ranged from 7.5 to 9.0.

Carbohydrate Content of pPPPBs

Neutral sugar content of exemplary isolated and purified pPPPBs (n=3 separate isolations) are shown in Table 13. The total neutral sugar content of the isolated pPPPBs and purified pPPPBs can be determined by the phenol-sulfuric acid method using glucose as a reference standard as described in Dubois et al., 1956. Briefly, the following stock solutions are prepared. A 4% phenol solution is prepared by adding 4 g of phenol to 100 ml of water and mixing until dissolved. Glucose standards are made from a stock solution of 1 mM glucose. Finally, a 98% concentrated stock of sulfuric acid is used for the assay. Sample solutions containing the pPPPBs are made in triplicate by adding 5 g of purified pPPPBs to 25 ml of 0.05 N HCl. Sample solutions are mixed for 30 min at 37° C. and 10 to 100 μl of each prepared sample is added to 400 μl of phenol, 2 ml of 98% H₂SO₄, mixed again and allowed to react for 60 min. The change in color can be measured at an optical density (O.D.) of 490 nm and the amount of carbohydrate determined using a standard curve for glucose. The neutral sugar content of exemplary isolated pPPPBs and purified pPPPBs samples ranges from 0.1-0.9% and 12-18%, respectively.

Phosphate Content of pPPPBs

Phosphate content of exemplary isolated and purified pPPPBs are shown in Table 13. The total phosphate content of isolated and purified pPPPBs are determined by the phosphovanadate method. The following solutions are prepared and used to determine the absorbance at 490 run of the complex formed between the phosphate, vanadate and molybdate when mixed with solutions containing phosphate. An ammonium vanadate solution is prepared by dissolving 1.25 g of ammonium vanadate in 250 ml of warm ddH₂O and 10 ml of concentrated nitric acid. The solution is mixed and brought to a final volume of 500 ml with ddH₂O. An ammonium molybdate solution is prepared by dissolving 25 g of ammonium molybdate in 400 ml of warm ddH₂O. The solution is mixed and brought to a final volume of 500 ml with ddH₂O. The ammonium molybdate solution is filtered prior to use. Solutions containing samples of the pPPPBs are prepared by adding 1 g of the pPPPBs to 200 ml of 2.5 M nitric acid warmed to 80° C. and mixed for 30 min. The volume is then brought to 1 L with the addition of dd H₂O.

Reactions are prepared with the addition of 10 ml each of ammonium vanadate, ammonium molybdate, and phosphate solutions (either unknown pPPPBs solution or a standard phosphate solution of potassium phosphate monobasic) to an empty flask, respectively. The final reaction volume is brought to 100 ml with dd H₂O. Flasks are mixed and the absorbance at 465 nm measured using a spectrophotometer. The phosphate concentration of the pPPPBs is determined using a standard curve prepared using known concentrations of phosphate. The phosphate content for samples of isolated and purified pPPPBs, as shown in Table 13, ranges from 34-38% and 10-15%, respectively.

Calcium Content of pPPPBs

Calcium content of the pPPPBs can be determined using several methods known to a person skilled in the art. Calcium content of the pPPPBs is measured from the CaCO₃ content following the precipitation of calcium in the pPPPBs using ammonium oxaloacetate and heating the precipitate to 525° C. for 2 h, where Ca²⁺+C₂₀ ₄+H₂O CaC₂₀ ₄ and then CaC₂O₄+heat=CaCO₃+CO. Calcium content of isolated and purified samples of the pPPPBs, as shown in Table 13, ranges from 20-24% and 1-2%, respectively.

Infared Spectrophotometry Analysis of pPPPBs

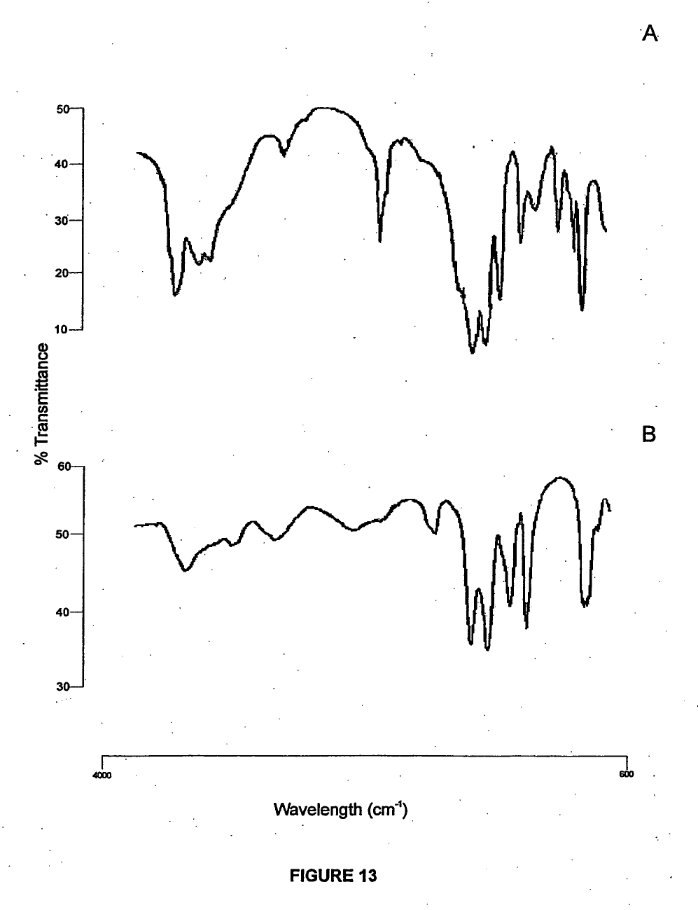

Interactions of the functional groups of isolated and purified samples of the pPPPBs respectively, as well as the nature of the bonding between the functional groups within each pPPPBs is determined using infrared spectroscopy. Representative IR spectra for purified and isolated pPPPBs are shown in FIG. 13(A) and (B), respectively. Representative samples of isolated and purified pPPPBs are prepared for IR spectroscopy as follows. Approximately 5-15 mg of the pPPPBs is pulverized and mixed with 1.5-2.0 g of potassium bromide (previously dried 5 h at 105° C.) with a motar and pestle. The powder is placed in a cylindrical stainless steel hydraulic press and compressed at a pressure between 1400 and 1762 kN/cm². IR spectra for the pPPPBs is determined between the wavelengths of 4000-600 cm.⁻¹.

### EXAMPLE III

### Measurements to Determine the Activity of pPPPBs

The biological activity of the pPPPBs can be determined using a variety of methods. One such assay to determine the activity of the pPPPBs is the rosette inhibition test as detailed in Morton et al. 1974 Nature 249(456):459-460. Briefly, blood samples are obtained from rabbits. Rabbit lymphocyte preparations are then obtained by density gradient centrifugation. Rabbit erythrocytes are washed 4 times in saline (2000 rpm/10 min) and suspended at a final concentration of 1% in phosphate buffered saline.

For determinations of the percentage of rabbit erythrocyte-rosette forming T-lymphocytes (R-RFT) in a sample, 0.25 ml of a prepared lymphocyte suspension (2.5×10⁶/ml) is incubated with 0.25 ml of the 1% rabbit erythrocyte suspension at 37° C. for 15 min, centrifuged at 800-1000 rpm for 5 min and maintained at 4° C. for 2 h, or overnight. The solution containing R-RFT is re-suspended, placed on a microscope slide and the number of R-RFT counted. Activated T-lymphocytes (i.e., having increased R-RFT capacity) are determined by mixing the test lymphocyte suspension with rabbit erythrocytes (and different concentrations of the pPPPBs as described below), immediately centrifuging for 5 min at 800-1000 rpm, re-suspending the cells, placing the cells on a microscope slide and counting the number of rosettes formed using a microscope. The rosette pattern criteria for activated T-lymphocytes is considered to be 3 or more rabbit erythrocytes adhered to a single T-lymphocyte.

In order to test the activity of the pPPPBs, separate solutions containing 0.25 ml, 1 ml and 4 ml of the pPPPBs are incubated with T-lymphocytes for 2 h at 37° C. Following this, rabbit erythrocytes are added to the above mixtures for 18 h at 2-8° C. Samples are centrifuged for 5 min at 800-1000 rpm, re-suspended in buffer, placed on microscope slides and the number of rosettes counted using a microscope. The rosette pattern criteria for activated T-lymphocytes are considered to be 3 or more erythrocytes adhered to a single T lymphocyte. A preparation of the pPPPBs is considered to be active if the number of activated T-lymphocytes forming rosette patterns is at least 2-fold higher than when compared to control levels, in the absence of the pPPPBs.

Another assay that can be used to characterize every newly synthesized batch of pPPPBs is to determine protein and carbohydrate content of isolated pPPPBs. The protein assay can be the modified Lowry assay and the carbohydrate assay can be the Dubois method, as described previously, however any protein or carbohydrate assay system familiar to a person skilled in the art may be used. An isolated batch of pPPPBs is considered to be active if the protein content of the isolated pPPPBs is higher than 0.1% of the total mass. In addition, an isolated batch of pPPPBs is considered to be active if the carbohydrate content of the isolated pPPPBs is higher than 0.1% of the total mass. If either % protein or % carbohydrate values are determined to be less than 0.1% of the total mass, the isolated pPPPBs are considered to be inactive.

### EXAMPLE IV

### Toxicity Studies

A measurement of the toxicity of pPPPBs can be determined using the LD₅₀ assay. Mice are injected with increasing concentrations of purified pPPPBs, and monitored for various biological responses including, but not limited to LD₅₀, feeding behavior and by monitoring the response to an immune challenge.

The pPPPBs are dissolved in sterile saline solution (0.9% NaCl) to the following concentrations: 1000 mg/ml (group 1); 100 mg/ml (group 2); 10 mg/ml (group 3); 1 mg/ml (group 4); and zero mg/ml (group 5). At the highest concentration, the white powder is difficult to dissolve and shows a mild precipitate that did not disappear completely, even with vigorous shaking.

Three month old male Balb/c mice with a weight of 20 g are used to measure the toxicity of the pPPPBs. Mice are maintained with food and water ad libitum throughout the experiment. Groups of six animals are used for each dose. Each mouse receives an injection of 0.1 ml of the pPPPBs in the left thigh, 3 times daily for 5 days. Animals in group 1 receive 300 mg of the pPPPBs per day, a dose 100 times the recommended estimated dosage for humans. The results of the study are that, 1) none of the mice showed any side effects from the pPPPBs; 2) feeding habits and behavior of the mice did not change following injection of the pPPPBs or throughout the study; and 3) no local reactions or deaths were observed as a result of the injections of the pPPPBs. These results show that the pPPPBs are non-toxic and safe to use on mammalian subjects.

### EXAMPLE V

### Effect of pPPPBs in Immune Compromised Mice and Rats Infected with Opportunistic Pathogens

A primary objective of this study is to analyze responses of immune system markers in immunocompromised mice and mice infected with opportunistic pathogens following treatment with the pPPPBs. Mice and rats under both experimental paradigms are injected with 300 mg/ml of the pPPPBs per day for the duration of all studies. Control animals are injected with saline solution. Techniques used herein to produce mice or rats that are immunocompromised and to infect mice with opportunistic pathogens are familiar to a person skilled in the art.

In one study, mice are given splenectomies in order to depress their immune system activity. The level of natural killer cells (NK cell) and macrophage activity fell dramatically in control animals as expected. Mice injected with the pPPPBs however, exhibit significant increases in the level of NK cell and macrophage activity. Levels of monocyte mobilization, polymorphonuclear cell activity, concentrations of cytokines IL-1 and INFγ as well as concentrations of the colony stimulating factors GM-CSF, G-CSF and M-CSF increase significantly over control animals that are not treated with the pPPPBs. Similarly, overall hematopoeitic activity is much higher in mice treated with the pPPPBs. There are no local reactions or deaths as a result of injections the pPPPBs. These results show that the pPPPBs can dramatically modulate immune system activity in immunocompromised mice.

In a second study, Sprague-Dawley rats are given splenectomies in order to depress their immune system activity and then infected with Pneumocistis carinii and the course of infection is followed. Rats treated with the pPPPBs as above, exhibit a significantly lower number of pulmonary cysts, a hallmark of P. carinii infection, significant increases in the level of NK cell and macrophage activity, monocyte mobilization, polymorphonuclear cell activity and concentrations of granulocyte-macrophage colony stimulating factor, GM-CSF, when compared to control animals treated with saline alone. There are no local reactions or deaths as a result of injections of the pPPPBs. These results show that the pPPPBs can dramatically modulate immune system activity in immunocompromised rats.

In a third study, immunocompromised mice having severely depressed immune systems are infected with either Candida albicans, Eshericheria coli, or Staphylococcus aureus, injected with the pPPPBs (or saline for control animals) and the course of the infection by these opportunistic pathogens is then followed. The survival rate of animals infected with any of the above pathogens is significantly enhanced when treated with pPPPBs. Saline treated animals succumb to infection much faster than pPPPB-treated mice.

Moreover, mice injected with pPPPBs exhibit significant increases in the level of NK cell and macrophage activity. Levels of monocyte mobilization, polymorphonuclear cell activity, concentrations of cytokines IL-1 and INFγ as well as concentrations of colony stimulating factors GM-CSF, G-CSF and M-CSF increase significantly over control animals that are not treated with the pPPPBs. Similarly, overall hematopoeitic activity is much higher in infected immunocompromised mice treated with the pPPPBs of the invention. Phagocytic activity of macrophages is measured as described below. Phagocytic activity increases dramatically in pPPPBs-treated mice and there are no local reactions or deaths as a result of injections of the pPPPBs. These results show that the pPPPBs are non-toxic and can dramatically modulate immune system activity in immunocompromised mice. are infected with opportunistic pathogens.

### EXAMPLE VI

### Clinical Trials in Patients Presenting with Traumatic Injuries

A primary objective of this study is to analyze the frequency of infections, the duration of the hospital stay, quantify the phagocytic index (as described above) and compare these results with those from patients in similar conditions but without the benefit of treatment with the pPPPBs. Secondary objectives are to observe whether upon treatment with the pPPPBs, patients have improved immune responses and clinical outcomes, and whether they accelerate quicker to a cured state and return to daily activities faster following their treatment for traumatic injury. This is a prospective experimental double blind and random study.

The participants are patients of the Balbuena Hospital for Urgencies (Hospital de Urgencias Balbuena de los Servicios Medicos del DDF.). Inclusion criteria for subjects are as follows: 15 to 65 years of age, either gender, not receiving immunosuppressive treatment, and healthy prior to trauma. Patients presenting with traumatic injury who did not comply with any of the above mentioned criteria are excluded from the protocol. Subjects are excluded from the study if they discontinue the study, transfer to another hospital, self-discharge from the hospital, or die within 24 h of being admitted.

Three groups are constituted each with at least 40 patients: a) patients with a compound fracture of a long bone, b) patients with a penetrating wound of the abdomen and/or thorax, and c) patients with a grade II or III head concussion.

The reasons for choosing these three groups are: a) to discriminate the effect of stress on the immune response in addition to an intense traumatic injury and surgery (patients with a compound fracture of a long bone always require decontaminating treatment with general anesthesia); b) stress in addition to a light traumatic injury or surgery and a certain alteration to homeostasis as the patient is able to ingest food and liquids orally (patients with a penetrating wound to the abdomen and/or thorax); and c) intense traumatic injury in addition to grave alteration of homeostasis but without stress (patients with a grade II and III head concussion).

In a double blind and random study having signed their previous informed consent in compliance with the Helsinki declaration, the patients are administered the pPPPBs (2 capsules of 500 mg orally every 8 hours) or a placebo (2 capsules orally every 8 hours) for the duration of their stay in hospital.

A blood specimen is then taken from each patient for the routine laboratory tests and an additional 10 ml in order to determine the phagocytic index. The phagocytic index is measured using the following method. Blood (total volume 10 ml) is placed in two separate test tubes. The first contains 7 ml and is used to obtain serum. The second test tube contains 3 ml of blood along with glass beads to remove the fibrin from the blood sample, termed defibrinated blood.

The phagocytic index is determined by placing 8 drops of defibrinated blood on 3 clean slides (previously treated with sulfuric acid and rinsed sequentially in running water and in double distilled water, ddH₂O). Slides containing defibrinated blood are then placed in a moist chamber for 30 min at 37° C. to adhere the phagocytes to the glass slides. Slides are then washed with saline solution. In a separate preparation, 0.5 ml of serum and 0.5 ml of yeast cells (1×10⁸ cells) are mixed and incubated at 37° C. for 20 min. The test tube containing the serum is centrifuged and the top layer discarded. The saponin treated yeast cells are suspended in 2 ml MEM (Minimum Essential Medium) and 0.5 ml Nitro Blue Tetrazolium (NBT). This solution is added to the cells on the slide which are incubated previously at 37° C. for 30 min and which contain the phagocytic blood cells. These slides are then washed with saline solution in petri dishes, stained with 0.5% safranine for 10 min at room temperature and rinsed in running water.

After drying and mounting with resin, the number of yeast cells that are contained within the phagocytic blood cells (phagocytosed yeast cells) are counted with the aid of a microscope. Data is organized into the following 5 categories: A) phagocytic blood cells which contain zero yeast cells, B) phagocytic blood cells which contain 1 to 2 yeast cells, C) phagocytic blood cells which contain 3 to 5 yeast cells, D) phagocytic blood cells which contain 6 to 9 yeast cells, and E) phagocytic blood cells which contain more than 10 phagocytosed yeast cells. Complete blood counts and blood chemistry analyses are performed and participating patients are clinically evaluated daily. The median and standard deviation are calculated for the reported data.

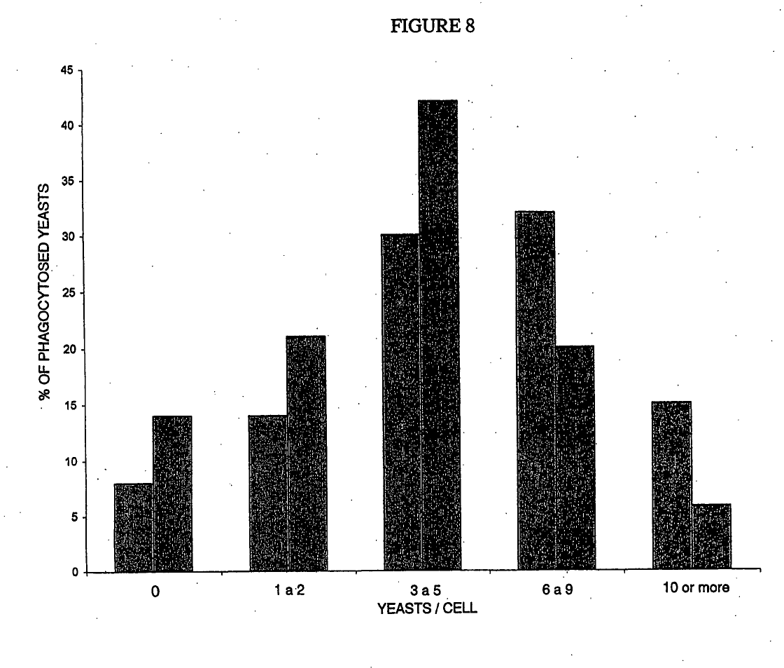

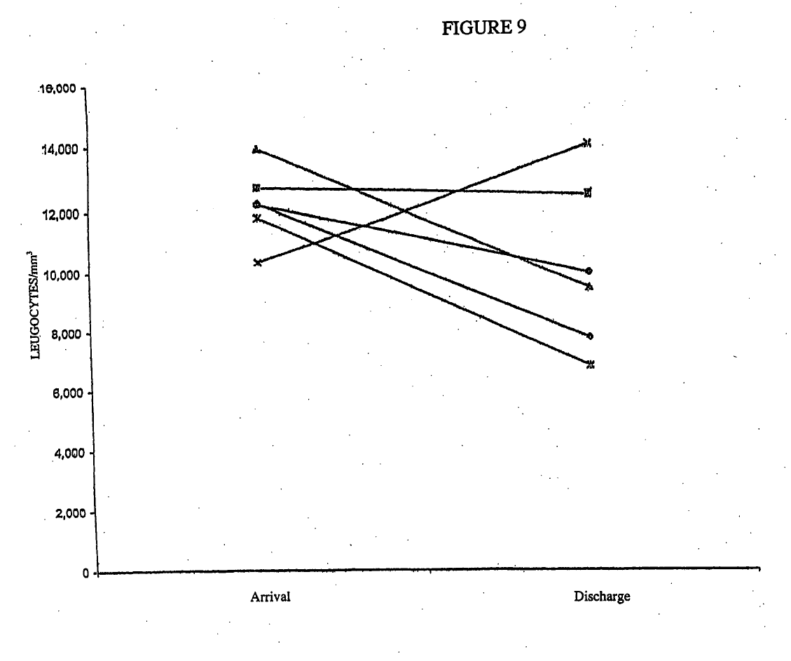

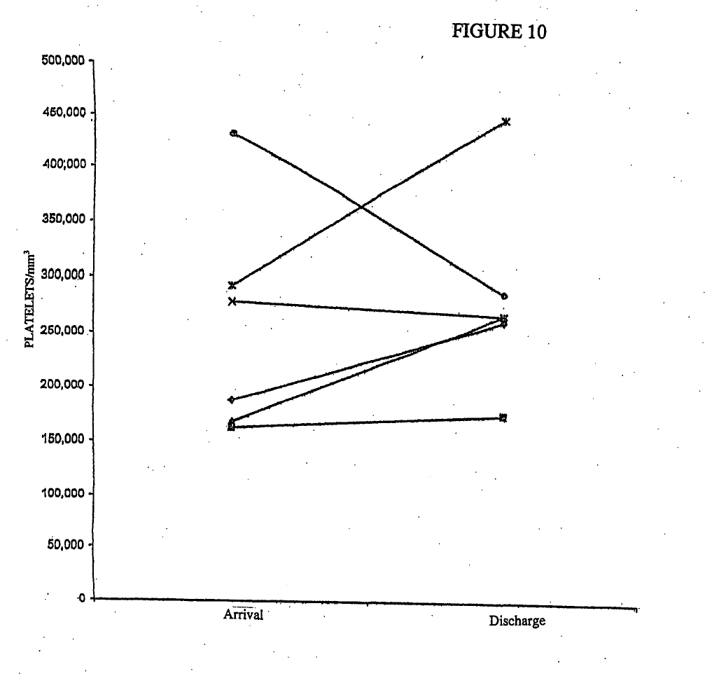

In patients with head concussion treated with the pPPPBs of this invention, a 3-fold increase (from 5% to 15%), as shown in FIG. 8, is observed in the number of cells with greatest phagocytic activity, category E above. In addition, those blood cells in category D which are isolated from patients receiving the pPPPBs show a modest increase in their measured phagocytic index increasing from 20% (placebo) to 32%. At the same time the blood cells with no phagocytic activity, category A, or reduced phagocytic activity, categories B and C, exhibit a decrease in their measured phagocytic index, from patients treated with the pPPPBs compared to those treated with placebo. The phagocytic index decreases from 14% to 8%, 21% to 14% and 42% to 30% for categories A, B and C, respectively (FIG. 8, Table 1). Upon the admission of patients presenting with grade II or III concussions, the number of leukocytes/ml of blood exceeds that seen in a normal population (FIG. 9, Tables 2, 3). However, these values did return to normal levels in patients treated with the pPPPBs when compared to those given the placebo (FIG. 9, Tables 2, 3).

In patients presenting with a compound fracture of a long bone and that are treated with the pPPPBs, there is again a 3-fold increase in the number of blood cells with greatest phagocytic activity, category E, from 1.5% (placebo) to 4.7% (treated). In addition, those blood cells in category D which are isolated from patients receiving the pPPPBs showed an increase in their measured phagocytic index increasing from 13% (placebo) to 29.5% (FIG. 6, Table 4). Concurrently the blood cells with no phagocytic activity, category A, or reduced phagocytic activity, categories B and C, exhibit a decrease or no change in their measured phagocytic index, from patients treated with the pPPPBs compared to those treated with placebo. The phagocytic index decreases from 40.6% to 37.3% for category C and remains unchanged for categories A and B (FIG. 6, Table 4). In patients presenting with a compound fracture of the long bones, the number of leukocytes remains at normal levels while the patients treated with the placebo show persistent leukocytosis (FIG. 9, Tables 5 and 6).

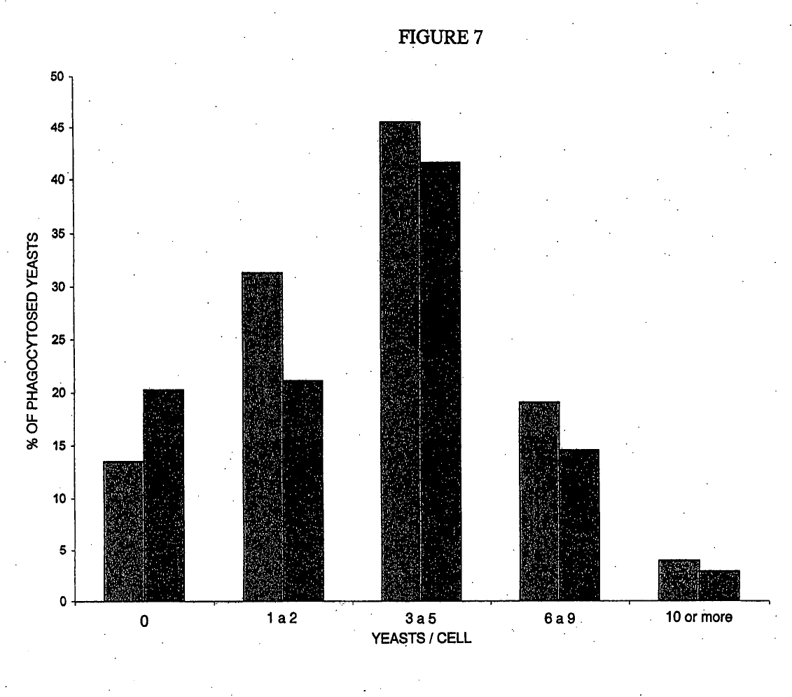

The results from patients presenting with penetrating wounds of the abdomen and/or thorax and treated with the pPPPBs show a very small increase in the number of blood cells with greatest phagocytic activity, category E, from 3% (placebo) to 4% (treated). In addition, those blood cells in category D which are isolated from patients receiving the pPPPBs show an increase in their measured phagocytic index increasing from 14% (placebo) to 19% (FIG. 7, Table 7). Isolated blood cells with no phagocytic activity, category A, exhibit a decrease in their measured phagocytic index, dropping from 20.2% in patients treated with the placebo to 13.5% in cells isolated from patients treated with the pPPPBs (FIG. 7, Table 7). The phagocytic index in blood cells with reduced phagocytic activity, categories B and C show a modest increase, from 21% (placebo) to 31.5% (treated) and 41.5% to 45.5%, respectively (FIG. 7, Table 7). In patients presenting with a penetrating wound of the abdomen and/or thorax the number of leukocytes/ml of blood remains at normal levels and is unchanged in patients treated with placebo (FIG. 9, Table 9). However, as seen in patients treated with the pPPPBs the number of leukocytes/ml decreases to normal levels following treatment (FIG. 9, Table 8).

The hematocrit (Hto%) and the level of hemoglobin (Hb%) in the blood taken from patients remains unchanged despite the type traumatic injury presented or the treatment given, placebo or pPPPBs, (Tables 2, 3, 5, 6, 8 and 9). The number of platelets/ml of blood drawn increases in patients from all three traumatic injury groups who are given the pPPPBs (Tables 2, 5 and 8). Contrary to this, patients given placebo show no change in the number of platelets/ml blood drawn despite the type of traumatic injury treated (Tables 3, 6 and 9).

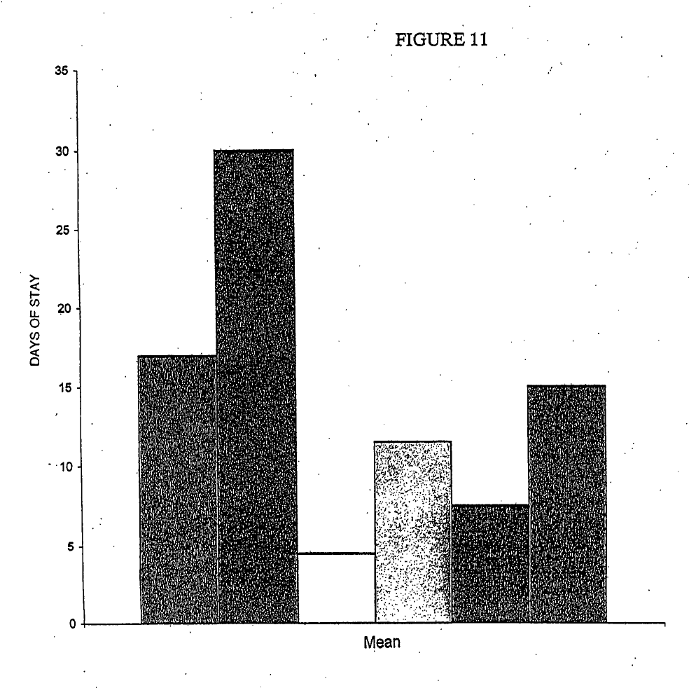

The hospital stay duration for patients presenting with grade II or III concussions is reduced from a mean of 30 days in patients treated placebo to almost half or 17 days in patients treated with the pPPPBs (FIG. 11, Table 10). The hospital stay duration for patients presenting with a compound fracture of a long bone is reduced more than 2-fold from a mean of 11.5 days in patients treated placebo to 4.5 days in patients treated with the pPPPBs (FIG. 11, Table 11). The hospital stay duration for patients presenting with a penetrating wound of the abdomen and thorax is reduced from a mean of 20.2 days in patients treated placebo to 13.5 days in patients treated with the pPPPBs (FIG. 11, Table 12). The range of hospital stay for patients treated with placebo is from 8 to 31 days and for those patients treated with the pPPPBs, the range decreases significantly to 3 to 12 days.

No adverse reactions or infections due to the use of the pPPPBs are observed in the patients treated with pPPPBs. Data shows that the pPPPBs improved the phagocytic index of treated patients. An improvement in this index shows that the ability of a patient's phagocytic blood cells to phagocytose (engulf) foreign cells or necrotic tissues increases in patients treated with the pPPPBs. An increase in the number of platelets/ml of blood (prohematopoietic) in patients is a finding that closely matches that seen in patients with cancer, but more studies are needed to analyze this activity in trauma patients. Principal effects in patients treated for all three types of traumatic injury that received the pPPPBs are a reduction in the duration of hospital stay, an improved clinical evolution and an absence of post-operative infections. Treatment with the pPPPBs will undoubtably result in significant economic savings in terms of medical treatment cost and length of hospital stay and improve the life of these patients.

### EXAMPLE VII

### Clinical Trials in Cancer Patients Presenting with Neutropenia following Chemotherapy

Neutropenia, the presence of an abnormally small number of neutrophil cells in the blood, is a common occurrence following chemotherapy treatment in cancer patients. Commonly, infections that threaten the life of the cancer patients are a direct result of chemotherapy-, and to a lesser extent radiation therapy-induced neutropenia. Approximately 90% of cancer patients present/display moderate to severe neutropenia following chemotherapy, causing further delays in treatment. In addition, the appearance of infections prolongs patient hospitalization.

A primary objective of this study is to analyze the utility of the pPPPBs in decreasing the moderate and severe neutropenia in adult cancer patients who have received chemotherapy as part of their cancer treatment. Secondary objectives are to observe whether, upon treatment with the pPPPBs, patients have improved immune responses and clinical outcomes, and whether they accelerate quicker to a cured state and return to daily activities faster following treatment. This is a prospective experimental double blind and random study.

Other criteria tested are whether 1) the recovery from neutropenia in adult cancer patients that underwent chemotherapy and are treated with pPPPBs is equal to the recovery in patients not treated with pPPPBs; 2) the recovery from neutropenia in adult cancer patients that underwent chemotherapy and are treated with the pPPPBs is better than the recovery in patients not treated with the pPPPBs; and 3) the number of infections that appear in cancer patients that underwent chemotherapy treatment and given the pPPPBs are less than the number of infections in patients not treated with the pPPPBs.

Participants in the study are patients at the Hospital Miguel Dorantes Meza, Xalapa Veracruz, Mexico. Inclusion criteria are as follows: cancer patients between the ages of 15 to 80 years of either gender, that are presenting with moderate and/or severe neutropenia with or without infection. Patients included in the study are diagnosed with the following cancers: ovarian, breast, lymphatic, rectal, colon, stomach, lung, kidney, cervical, bone as well as abdominal and sinovial sarcomas. Cancer patients presenting with little or no neutropenia following chemotherapy who did not comply with any of the above mentioned criteria are excluded from the protocol. Subjects are also excluded from the study if they discontinue the study, transfer to another hospital or self-discharge from the hospital.

One hundred (100) adult cancer patients who presented with neutropenia following a previous chemotherapy treatment are enrolled in this study. In a double blind and random study having signed their previous informed consent in compliance with the Helsinki declaration, 57 patients are administered the pPPPBs orally, 1 g every 8 h, for a period of 21 days following each cycle of chemotherapy, 43 patients are administered a placebo under the same dosing regimen. A blood specimen is taken twice daily from each patient for routine laboratory blood work tests including the determination of percent hemoglobin, hematocrit, as well as the number of erythrocytes, platelets, leukocytes, neutrophils, lymphocytes, monocytes, eosinophils and basophils in treated versus control patients. The median and standard deviation are calculated for all reported data. Every three (3) days in the morning and night a determination of liver and renal function is performed along with the measurement of hematological parameters to ensure that the condition of all patients remains stable throughout the study.

Table 14 shows hematological data from cancer patients that underwent no chemotherapy treatment (G0-control) and either 1 (G1), 2 (G2), 3 (G3) or 4 (G4) chemotherapy treatments, respectively. Data show that hematological parameters for patients that received 4 chemotherapy treatments are significantly lower than control (Table 14). The hematological parameters including, blood hematocrit, levels of hemoglobin and most importantly the number of neutrophils, remains constant in cancer patients treated with the pPPPBs following chemotherapy and are unchanged when compared to controls, even after 4 chemotherapy/pPPPBs treatment cycles (Table 15).

No adverse reactions or infections due to the use of the pPPPBs are observed in the patients treated with the pPPPBs. Data show that the pPPPBs improved all hematological indexes of treated patients undergoing chemotherapy versus those undergoing chemotherapy alone. There are no observable side effects in patients treated with pPPPBs indicating that the pPPPBs of this invention are non-toxic. Principal effects in cancer patients treated with chemotherapy that received the pPPPBs are a reduction in the duration of the stay in the hospital, an improved clinical evolution, a clear reduction in the cases of neutropenia and significantly reduced opportunistic infections.

### EXAMPLE VIII

### Clinical Trials in Patients Presenting with Breast Cancer-Study I

A primary objective of this study is to analyze the utility of the pPPPBs in decreasing the incidents of myelosuppression, the inhibition of bone marrow function, in adult breast cancer patients who have received multiple chemotherapy treatments as part of their cancer therapy. Secondary objectives are to observe whether upon treatment with the pPPPBs, pPPPBs-treated patients have improved immune responses and clinical outcomes, and whether they accelerate quicker to a cured state and return to daily activities faster following treatment. This is a prospective experimental double blind and random study.

Participants in the study are patients treated at the Mexican National Cancer Institute, Mexico City, Mexico. Inclusion criteria are as follows: breast cancer patients, female between the ages of 27 to 74 years, that are presenting with mammary adenocarcinoma as reported using histological methods familiar to a person skilled in the art, a Karnofsky score of between 80-100% and have not previously received chemotherapy, radiation therapy, or hormone replacement therapy. Cancer patients treated using any of the previously mentioned three treatments or a combination thereof before entering the study, are excluded. Subjects are excluded from the study if they discontinue the study, transfer to another hospital or self-discharge from the hospital.

Thirty-six (36) patients who are diagnosed with breast cancer are enrolled in the study. In a double blind and random study having signed their previous informed consent in compliance with the Helsinki declaration, 18 patients, average age 46.8 years, are administered the pPPPBs, orally, 500 mg every 12 h, for a period of 21 days along with a standard chemotherapy treatment. Chemotherapy treatment is run for the same 21 day period using the following dosing regimen: 2×500 mg of 5-fluorouracil on day 1, 8 and 21; 2×35 mg of methotrexate on day 1, 8 and 21; and 2×500 mg of cyclophosphamide on day 1 and 21. Eighteen (18) patients, average age 48.7 years, are administered a placebo in place of the pPPPBs under the same dosing regimen, plus the standard chemotherapy treatment described above. A blood sample is taken twice daily from each patient for routine laboratory blood work tests including the determination of percent hemoglobin, hematocrit, as well as the number of erythrocytes, platelets, leukocytes, neutrophils, lymphocytes, monocytes, eosinophils and basophils in treated versus control patients. In addition, a bone marrow sample is taken from each patient at the beginning and end of the treatment cycle. Data is used to determine the effect of the pPPPBs on the occurrences of myelosuppression, anemia due to decreased hemoglobin, leukopenia, neutropenia and thrombocytopenia.

The following are the reported results of the study. A lower number of leukocytes, neutrophils, platelets and percentage of hemoglobin in the blood are observed in all patients. Three cases of leukopenia are present in 17% (3/18) of patients in the treatment group that received the pPPPBs and each of these patients has a leukocyte count of less than 3.0×10³/ml (3/18=17%), as compared to a normal healthy population of individuals. The number of leukocytes in each of these patients is 2.5×10³/ml ,2.7×10³/ml and 2.9×10³/ml, respectively. A decrease in the number of platelets, less than 100×10³/ml, is observed in 28% (5/18) of the patients that received treatment of the pPPPBs in addition to chemotherapy, ranging from 56-76×10³/ml. Most patients show little or no change in level of their hematopoietic status when given the pPPPBs, during their chemotherapy treatment. In addition, there is a much faster recovery rate from leukopenia in cancer patients given the pPPPBs compared to those given placebo, indicating that the pPPPBs significantly increases the recovery rate from chemotherapy and improves patient outcome. There is a significant decrease in the hematopoeitic status of patients given placebo plus chemotherapy treatment as indicated by the data showing that 66.6% of these patients have a neutrophil count of less than 2×10³/ml, compared to 27.7% of patients given daily doses of the pPPPBs during chemotherapy treatment. Thus, there is a significant. increase in the occurrences of anemia, leukopenia and neutropenia in patients not given the pPPPBs. There is no change in the number of platelets/ml of blood between either treatment group. Interestingly, in the group that received the pPPPBs there are 5 patients that exhibit a reduction in the size of their tumor by more than 50%, as measured by tumor volume.

No adverse reactions or infections due to the use of the pPPPBs are observed in any of the patients treated with the pPPPBs. Data show that the pPPPBs improves all hematological indexes of treated patients undergoing chemotherapy versus those undergoing chemotherapy alone. There are no side effects in patients treated with pPPPBs, indicating that the pPPPBs of this invention are non-toxic. Principal effects in breast cancer patients treated with chemotherapy and treatment of the pPPPBs are an improvement in clinical evolution, a clear reduction in the cases of neutropenia and leukopenia and significantly reduced opportunistic infections as a result of the chemotherapy-induced depression of the patients' immune systems.

### EXAMPLE IX

### Clinical Trials in Patients Presenting with Breast Cancer-Study II

A second study at the National Cancer Institute in Mexico City, Mexico using the experimental criteria as found in the above Example VIII has been completed.

Forty-six (46) patients who are diagnosed with breast cancer are enrolled in the study. In a double blind and random study having signed their previous informed consent in compliance with the Helsinki declaration, 26 patients are administered the pPPPBs, orally, 500 mg every 12 h, for a period of 21 days along with a standard chemotherapy treatment. Chemotherapy treatment is run for the same 21 day period using the following dosing regimen: 2×500 mg of 5-fluorouracil on day 1, 8 and 21; 2×35 mg of methotrexate on day 1, 8 and 21; and 2×500 mg of cyclophosphamide on day 1 and 21. Twenty (20) patients are administered a placebo in place of the pPPPBs under the same dosing regimen, plus the standard chemotherapy treatment described above. A blood sample is taken twice daily from each patient for routine laboratory blood work tests including the determination of percent hemoglobin, hematocrit, as well as the number of erythrocytes, platelets, leukocytes, neutrophils, lymphocytes, monocytes, eosinophils and basophils in treated versus control patients. In addition, a bone marrow sample is taken from each patient at the beginning and end of the treatment cycle. Data is used to determine the effect of the pPPPBs on the occurrences of leukopenia, medular hypoplasia of bone marrow and thrombocytopenia.

Results of the study show that a lower number of leukocytes, neutrophils, platelets and percentage of hemoglobin in the blood are observed in all patients enrolled in the study. In patients that received treatment with the pPPPBs, medular hypoplasia of the bone marrow is found in only 26% (12/46) patients which is significantly lower than the 65% (13/20) in the control group treated with placebo. In addition, no patients that receive the pPPPBs develop thrombocytopenia (0/46) and the time of recovery from leukopenia although rare in these patients, 11% (5/46) is much faster. In contrast, the placebo group patients exhibit both thrombocytopenia and leukopenia and their recovery time from a cycle of chemotherapy is much longer. No adverse reactions or infections due to the use of the pPPPBs are observed in the patients treated with the pPPPBs. Data show that the pPPPBs improves all hematological indexes of treated patients undergoing chemotherapy and the pPPPBs treatment versus those undergoing chemotherapy, but not given the pPPPBs. There are no side effects in patients treated with the pPPPBs, indicating that the pPPPBs of this invention are non-toxic. Principal effects in breast cancer patients treated with chemotherapy that received the pPPPBs are an improvement in clinical evolution, a clear reduction in the cases of thrombocytopenia, leukopenia, and bone marrow medular hypoplasia as well as significant reductions in opportunistic infections as a result of the chemotherapy-induced immune suppression.

### EXAMPLE X

### Clinical Trials in Patients Presenting with Small-Cell Carcinoma of the Lung

A primary objective of this study is to analyze the utility of the pPPPBs in decreasing the incidents of myelosuppression, the inhibition of bone marrow function, in adult small-cell carcinoma of the lung who have received multiple chemotherapy treatments as part of their cancer therapy. Secondary objectives are to observe whether upon treatment with the pPPPBs, patients have improved immune responses and clinical outcomes, and whether they accelerate quicker to a cured state and return to daily activities faster following treatment. This is a prospective experimental double blind and random study.

Participants in the study are patients treated at the Mexican National Cancer Institute, Mexico City, Mexico. Inclusion criteria are as follows: patients presenting with small-cell carcinoma of the lung, either gender, between the ages of 18 to 80 years, and whom have not previously received chemotherapy. Cancer patients treated with chemotherapy prior to the start date of the study are excluded. Subjects are also excluded from the study if they discontinue the study, transfer to another hospital or self-discharge from the hospital.

Forty (40) patients who are diagnosed with small-cell carcinoma of the lung are enrolled in the study. In a double blind and random study having signed their previous informed consent in compliance with the Helsinki declaration, 30 patients are administered the pPPPBs, orally, 500 mg every 12 h, for a period of 21 days along with a standard chemotherapy treatment. Chemotherapy treatment is run for the same 21 day period using the following dosing regimen: 2×120 mg of cisplatin on day 1, 8 and 21; 2×10 mg of mitocin C on day 1, 8 and 21; and 2×3 mg of vindesine on day 1 and 21. Ten (10) patients are administered a placebo in place of the pPPPBs under the same dosing regimen, plus the standard chemotherapy treatment described above. A blood sample is taken twice daily from each patient for routine laboratory blood work tests including the determination of percent hemoglobin, hematocrit, as well as the number of erythrocytes, platelets, leukocytes, neutrophils, lymphocytes, monocytes, eosinophils and basophils in treated versus control patients. In addition, a bone marrow sample is taken from each patient at the beginning and end of the treatment cycle. Data is used to determine the effect of the pPPPBs on the occurrences of myelosuppression and leukopenia.

The following are the reported results of the study. A lower number of leukocytes, neutrophils, platelets and percentage of hemoglobin in the blood are observed in all patients. The number of leukocytes in patients treated with the pPPPBs is an average of 3.05×10³/ml which is 83% of the normal level found in healthy blood donors with no evidence of cancer. There is no decrease in the number of platelets in any of the 30 patients treated with the pPPPBs. Most patients show little or no change in the level of their hematopoietic status when given the pPPPBs, during their chemotherapy treatment. In addition, there is a much faster recovery rate from leukopenia in cancer patients given the pPPPBs compared to those given placebo. This indicates that the pPPPBs significantly increases the recovery rate from chemotherapy and improves patient outcome in patients with small-cell carcinoma of the lung. There is a significant decrease in the hematopoeitic status of patients given placebo plus chemotherapy treatment as indicated by the data showing that 60% of these patients have a neutrophil count of less than 2×10³/ml. Thus, there is a significant increase in the occurrences of leukopenia in patients not given the pPPPBs. There is no change in the number of platelets/ml of blood or the level of medular hypoplasia in the bone marrow between either treatment group.

No adverse reactions or infections due to the use of the pPPPBs are observed in the patients treated with the pPPPBs. Data show that the pPPPBs improved all hematological indexes of treated patients undergoing chemotherapy versus those undergoing chemotherapy with no additional treatment. There are no side effects in patients receiving pPPPB treatment, indicating the pPPPBs of this invention are non-toxic. Principal effects in patients with small-cell carcinoma of the lung treated with chemotherapy and that receive the pPPPBs are, an improved clinical evolution, a clear reduction in the cases of leukopenia and significantly reduced opportunistic infections as a result of the chemotherapy-induced depression of the patients' immune systems. Treatment with the pPPPBs will undoubtably result in significant improvement in the quality of life of these cancer patients.

### EXAMPLE XI

### Clinical Trials using pPPPBs in Cancer Patients Following Surgery to Remove Tumors

In cancer patients, any type of surgery is “delicate”, especially in patients that are immunocompromised due to chemotherapy or radiation therapy. Any infections that may occur following a surgical procedure, to remove a tumor for example, may lead to dramatic imbalances in the overall homeostasis of a cancer patient. Therefore, the progress of the healing process must be carefully monitored in cancer patients recovering from surgery. The healing of any wound or injury, is a complex series of processes with interdependent stages that control a number of biochemical responses at the cellular level. However, despite advances in the treatment and diagnosis of cancers and in the surgical procedures used to treat different cancers, the healing of postoperative wounds continues to be a large clinical problem for these patients. Immunocompromised patients may be susceptible to infection from a number of sources including unsterile surgical instruments or from the use of ventilators and catheters. Therefore, a need remains for an effective treatment to assist in the wound healing process that has little or no toxic effect on patients while at the same time stimulates the immune system, especially in cancer patients who have been treated with radiation or chemotherapy.

The pPPPBs are used in the following clinical trial in order to determine the possibility of using this compound to facilitate the healing process in cancer patients by increasing the actions of macrophages and polymorphonuclear cells that modulate the inflammatory process during wound healing.

A primary objective of the study is to evaluate the ability of the pPPPBs to potentiate the healing process, reduce the time of hospitalization and the occurrence of infections in post-operative cancer patients. Other objectives include 1) directly demonstrate in these patients, protection of the immune system during the healing process; 2) demonstrate the prophylaxis and attenuation of infections by comparison of the results with patients who did not receive the pPPPBs; 3) evaluate the cost benefit relationship of the pPPPBs treatment that occurs in relation to duration of hospital stay; 4) evaluate the immune state of these patients by means of examining hematological data collected from all patients in the study; 5) explore the possibility of “accelerated” healing with the use of the pPPPBs by monitoring the healing process of all patients following surgery; and 6) determine the number and type of opportunistic infections when they occur and the treatment used to ameliorate them. This is a prospective experimental double blind and random study.

Participants in the study are patients treated at the Hospital Miguel Dorantes Meza, Xalapa Veracruz, Mexico. Inclusion criteria are as follows: cancer patients between the ages of 18 and 60 years, with good nutritional health, a Karnofsky score of between 60-100%, a life expectancy of greater than 3 months, patients that will be undergoing radical surgery including either mastectomy, gastrecomy, colectomy, lobectomy, surgery of the head or neck, surgery of the genitals and feminine reproductive tract, prostatectomy, partial hepatectomy or surgery of the bile duct. Patients are excluded from the study if they where under 18 years or over 60 years, given corticosteriods or other immunomodulators, such as levamisol, less than 3 months before surgery, immunodepressed due to chemotherapy or radiation treatment prior to the study, discontinue the study, transfer to another hospital or self-discharge from the hospital.

One hundred (100) adult patients of both sexes, diagnosed with various cancers are enrolled in this study. In a double blind and random study having signed their previous informed consent in compliance with the Helsinki declaration, 45 patients are administered the pPPPBs orally, 6×500 mg tablets daily five (5) days prior to their surgery. Following surgery these 45 patients are to continue taking 6×500 mg doses per day for 10 days after the operation. Fifty-five (55) patients are administered a placebo in place of the pPPPBs under the same dosing regimen. A blood sample is taken twice daily from each patient for routine laboratory blood work tests including the determination of percent hemoglobin, hematocrit, as well as the number of platelets, leukocytes and neutrophils in treated versus control patients. In addition, all patients are monitored for post-operative pain and the state of external scarring by daily examination of the wound site. In particular, the edges of the wound, the stability of the sutures, and signs of inflammation including reddening and swelling are carefully monitored.

The following are the reported results of the study. Overall a lower incidence of opportunistic infection and post-operative infection occur in patients given treatment of the pPPPBs compared to placebo alone. Most patients (95%) show little or no change in the level of their hematopoietic status and have no infections at the wound site when given the pPPPBs following surgery. Contrary to this, patients not receiving treatment with pPPPBs exhibit increased occurrences of opportunistic infection following surgery. Recovery rate is significantly accelerated in 80% (36/45) of the patients given pPPPBs compared to those given placebo. Finally, patients that are administered the pPPPBs exhibit little or no pain compared to patients given the placebo. This indicates that the pPPPBs significantly increase the recovery rate from radical surgery and improves patient outcome. There was no change in the average number of platelets, leukocytes or neutrophils or the % hemoglobin in patients administered the pPPPBs, after surgery when compared to the same parameters monitored 5 days prior to surgery (Table 16).

No adverse reactions or infections due to the use of the pPPPBs are observable in patients treated with the pPPPBs. Data show that the pPPPBs improves all hematological indexes of treated patients undergoing radical surgery to remove tumors versus those undergoing radical surgery but not given the pPPPBs. There are no side effects in patients treated with pPPPBs indicating that the pPPPBs of this invention are non-toxic and safe to use on human subjects. Principal effects in patients that underwent radical surgical procedures and that received the pPPPBs are, an improved clinical evolution, a clear reduction in the cases of leukopenia and significantly reduced opportunistic infections that may result from surgery.

From the foregoing description, one skilled in the art can easily ascertain the essential characteristics of this invention, and without departing from the spirit and scope thereof, can make various changes and modifications to the embodiments of the invention for adaptation to various usages and conditions. Such changes and modifications are properly, equitably, and intended to be within the full range of equivalence of the following claims.

TABLE 1
PHAGOCYTIC INDEX OF PATIENTS
WITH A HEAD CONCUSSION

| Yeast/Cells | pPPPBs (%) | Placebo (%) |
|---|---|---|
| 0 | 8 | 14 |
| 1-2 | 14 | 21 |
| 3-5 | 30 | 42 |
| 6-9 | 32 | 20 |
| more than 10 | 15 | 5 |

TABLE 2
EFFECT OF pPPPBs ON THE HEMATIC ANALYSIS IN PATIENTS
WITH A HEAD CONCUSSION
PATIENT TREATED WITH THE pPPPB

| | | | | |
|---|---|---|---|---|
| | | | Leukocytes | Platelets × |
| Number | Hto (%) | Hb. (%) | (/ml) | 10³ (/ml) |

AT ADMISSION

| | | | | |
|---|---|---|---|---|
| 1 | 46.6 | 15.5 | 10 600 | 184 |
| 2 | 42 | 15.2 | 20 800 | 217 |
| 3 | 40.1 | 14.8 | 10 300 | 176 |
| 4 | 44.1 | 116.2 | 11 000 | 156 |
| 5 | 45.3 | 16.5 | 15 700 | 263 |
| 6 | 40.2 | 14.8 | 4 600 | 170 |
| 7 | 37.3 | 13.6 | 11 400 | 232 |
| 8 | 36.9 | 13.6 | 8 100 | 167 |
| 9 | 24.3 | 8.7 | 16 200 | 120 |
| 10 | 39.4 | 14.0 | 13 700 | 186 |
| x | 39.62 | 14.29 | 12 240 | 177.1 |

AT RELEASE

| | | | | |
|---|---|---|---|---|
| 1 | 45.0 | 15.9 | 8 500 | 260 |
| 2 | 43.9 | 15.0 | 9 700 | 290 |
| 3 | 44.3 | 14.7 | 9 000 | 230 |
| 4 | 41.0 | 15.0 | 9 300 | 252 |
| 5 | 46.0 | 15.5 | 7 200 | 292 |
| 6 | 44.7 | 15.9 | 5 700 | 232 |
| 7 | 44.0 | 15.0 | 8 900 | 252 |
| 8 | 39.0 | 13.6 | 5 500 | 257 |
| 9 | 38.0 | 13.0 | 8 600 | 250 |
| 10 | 44.7 | 14.9 | 5 200 | 299 |
| x | 43.06 | 13.8 | 7 760 | 261.4 |

TABLE 3
EFFECT OF pPPPBs ON THE HEMATIC ANALYSIS IN PATIENTS
WITH A HEAD CONCUSSION
PATIENT TREATED WITH THE PLACEBO

| | | | | |
|---|---|---|---|---|
| | | | Leukocytes | Platelets × |
| Number | Hto (%) | Hb. (%) | (/ml) | 10³ (/ml) |

AT ADMISSION

| | | | | |
|---|---|---|---|---|
| 1 | 26.8 | 9.1 | 5 500 | 159 |
| 2 | 33.4 | 10.8 | 10 900 | 116 |
| 3 | 42.5 | 15.2 | 5 900 | 141 |
| 4 | 42.8 | 15.5 | 22 600 | 219 |
| 5 | 38.1 | 14.1 | 15 200 | 72 |
| 6 | 38.0 | 13.5 | 13 000 | 186 |
| 7 | 41.3 | 15.3 | 5 700 | 154 |
| 8 | 36.0 | 10.7 | 21 300 | 171 |
| 9 | 33.9 | 9.1 | 18 100 | 177 |
| 10 | 35.5 | 13.3 | 8 800 | 232 |
| x | 36.8 | 11.7 | 12 700 | 162.7 |

AT RELEASE

| | | | | |
|---|---|---|---|---|
| 1 | 26.8 | 9.1 | 5 500 | 159 |
| 2 | 37.0 | 12.3 | 13 200 | 159 |
| 3 | 41.2 | 13.1 | 9 500 | 144 |
| 4 | 43.6 | 13.6 | 15 000 | 231 |
| 5 | 38.8 | 14.8 | 8 700 | 262 |
| 6 | 40.0 | 13.3 | 13 700 | 161 |
| 7 | 42.9 | 14.3 | 11 000 | 175 |
| 8 | 34.0 | 13.0 | 14 000 | 209 |
| 9 | 25.1 | 10.1 | 12 000 | 218 |
| 10 | 39.0 | 13.0 | 9 800 | 149 |
| x | 36.8 | 12.6 | 11 240 | 186.7 |

TABLE 4
PHAGOCYTIC INDEX OF PATIENTS WITH AN
EXPOSED FRACTURE OF THE LONG BONES

| Yeast/Cells | pPPPBs (%) | Placebo (%) |
|---|---|---|
| 0 | 17 | 16.5 |
| 1-2 | 31 | 29 |
| 3-5 | 37.3 | 40.6 |
| 6-9 | 29.5 | 13 |
| more than 10 | 4.7 | 1.5 |

TABLE 5
EFFECT OF pPPPBs ON THE HEMATIC ANALYSIS OF PATIENTS
WITH AN EXPOSED FRACTURE OF THE LONG BONES
PATIENT TREATED WITH THE pPPPB

| | | | | |
|---|---|---|---|---|
| | | | Leukocytes | Platelets × |
| Number | Hto (%) | Hb. (%) | (/ml) | 10³ (/ml) |

AT ADMISSION

| | | | | |
|---|---|---|---|---|
| 1 | 33 | 13.1 | 15 900 | 182 |
| 2 | 33.9 | 12.5 | 18 100 | 154 |
| 3 | 40.8 | 16.2 | 15 000 | 190 |
| 4 | 38.4 | 13.8 | 7 800 | 170 |
| 5 | 35.1 | 14 | 12 700 | 149 |
| 6 | 37.4 | 14 | 13 300 | 106 |
| 7 | 25.8 | 15.9 | 17 000 | 210 |
| 8 | 23.4 | 12.4 | 13 300 | 180 |
| 9 | 20.8 | 11.2 | 10 600 | 142 |
| 10 | 28.4 | 15.3 | 15 300 | 197 |
| x | 31.2 | 13.9 | 13 900 | 168 |

AT RELEASE

| | | | | |
|---|---|---|---|---|
| 1 | 20.6 | 7.5 | 9 900 | 241 |
| 2 | 18.4 | 6.9 | 16 500 | 251 |
| 3 | 34.2 | 9.3 | 9 900 | 205 |
| 4 | 31.3 | 10.5 | 15 700 | 160 |
| 5 | 31.8 | 10.2 | 5 400 | 263 |
| 6 | 24.7 | 9.9 | 5 200 | 299 |
| 7 | 13.5 | 7.0 | 9 000 | 282 |
| 8 | 17.7 | 6.5 | 8 500 | 236 |
| 9 | 21.3 | 8.7 | 7 300 | 257 |
| 10 | 23.5 | 6.0 | 7 600 | 279 |
| x | 23.7 | 8.1 | 9 500 | 247.3 |

TABLE 6
EFFECT OF pPPPBs ON THE HEMATIC ANALYSIS OF PATIENTS
WITH AN EXPOSED FRACTURE OF THE LONG BONES
PATIENT TREATED WITH THE PLACEBO

| | | | | |
|---|---|---|---|---|
| | | | Leukocytes | Platelets × |
| Number | Hto (%) | Hb. (%) | (/ml) | 10³ (/ml) |

AT ADMISSION

| | | | | |
|---|---|---|---|---|
| 1 | 28.5 | 10.8 | 8 200 | 286 |
| 2 | 34.5 | 12.2 | 10 900 | 200 |
| 3 | 14.4 | 6.8 | 15 900 | 189 |
| 4 | 30.3 | 10.8 | 9 100 | 374 |
| 5 | 36.6 | 11.6 | 8 200 | 384 |
| 6 | 39.9 | 13.7 | 10 900 | 224 |
| 7 | 45.3 | 10.9 | 11 300 | 188 |
| 8 | 35.0 | 13.7 | 10 900 | 185 |
| 9 | 35.6 | 12.5 | 7 500 | 167 |
| 10 | 32.2 | 11.7 | 10 600 | 281 |
| x | 33.2 | 11.4 | 10 350 | 277.8 |

AT RELEASE

| | | | | |
|---|---|---|---|---|
| 1 | 36.1 | 12.7 | 7 900 | 131 |
| 2 | 30.5 | 9.8 | 9 500 | 334 |
| 3 | 38.2 | 11.8 | 16 900 | 444 |
| 4 | 33.5 | 11.6 | 8 700 | 233 |
| 5 | 40.2 | 15.4 | 15 700 | 230 |
| 6 | 37.9 | 11.7 | 17 900 | 254 |
| 7 | 39.9 | 15.4 | 18 500 | 321 |
| 8 | 32.7 | 17.8 | 15 400 | 143 |
| 9 | 33.7 | 11.7 | 13 800 | 352 |
| 10 | 36.7 | 14.3 | 17 700 | 331 |
| x | 35.9 | 13.2 | 14 200 | 267 |

TABLE 7
PHAGOCYTIC INDEX OF PATIENTS WITH A PENETRATING
WOUND OF THE THORAX AND/OR ABDOMEN

| Yeast/Cells | pPPPBs (%) | Placebo (%) |
|---|---|---|
| 0 | 13.5 | 20.2 |
| 1-2 | 31.5 | 21 |
| 3-5 | 45.5 | 41.5 |
| 6-9 | 19 | 14 |
| more than 10 | 4 | 3 |

TABLE 8
EFFECT OF pPPPBs ON THE HEMATIC ANALYSIS OF PATIENTS
PRESENTING WITH A PENETRATING WOUND OF
THE THORAX AND/OR ABDOMEN
PATIENT TREATED WITH THE pPPPB

| | | | | |
|---|---|---|---|---|
| | | | Leukocytes | Platelets × |
| Number | Hto (%) | Hb. (%) | (/ml) | 10³ (/ml) |

AT ADMISSION

| | | | | |
|---|---|---|---|---|
| 1 | 29.9 | 10.4 | 4 200 | 321 |
| 2 | 26.4 | 4.3 | 18 900 | 206 |
| 3 | 46.4 | 16.5 | 7 300 | 182 |
| 4 | 38.8 | 13.4 | 8 900 | 187 |
| 5 | 26 | 8 | 9 700 | 501 |
| 6 | 29.3 | 10.4 | 24 400 | 427 |
| 7 | 39.5 | 14.9 | 10 400 | 177 |
| 8 | 30.5 | 11.5 | 10 600 | 193 |
| 9 | 41.6 | 12.4 | 12 200 | 446 |
| 10 | 29.7 | 11.2 | 11 400 | 574 |
| x | 33.8 | 11.3 | 11 800 | 292.4 |

AT RELEASE

| | | | | |
|---|---|---|---|---|
| 1 | 31.3 | 11.1 | 6 500 | 372 |
| 2 | 29.7 | 7.4 | 8 900 | 308 |
| 3 | 45.7 | 15.9 | 5 200 | 400 |
| 4 | 38.0 | 13.5 | 5 100 | 391 |
| 5 | 30.2 | 10.6 | 7 600 | 506 |
| 6 | 30.7 | 10.8 | 12 500 | 661 |
| 7 | 31.6 | 13.8 | 5 300 | 350 |
| 8 | 30.6 | 11.8 | 5 000 | 320 |
| 9 | 32.5 | 12.4 | 6 600 | 532 |
| 10 | 28.7 | 11.7 | 5 300 | 326 |
| x | 32.9 | 11.9 | 6 800 | 446.5 |

TABLE 9
EFFECT OF pPPPBs ON THE HEMATIC ANALYSIS OF PATIENTS
PRESENTING WITH A PENETRATING WOUND OF
THE THORAX AND/OR ABDOMEN
PATIENT TREATED WITH THE PLACEBO

| | | | | |
|---|---|---|---|---|
| | | | Leukocytes | Platelets × |
| Number | Hto (%) | Hb. (%) | (/ml) | 10³ (/ml) |

AT ADMISSION

| | | | | |
|---|---|---|---|---|
| 1 | 13.8 | 4.3 | 5 400 | 56 |
| 2 | 33.6 | 12.1 | 8 200 | 134 |
| 3 | 28.5 | 10.0 | 15 700 | 96 |
| 4 | 23.5 | 8.7 | 13 100 | 164 |
| 5 | 49.9 | 15.7 | 8 900 | 262 |
| 6 | 30.2 | 10.1 | 13 000 | 636 |
| 7 | 20.3 | 8.7 | 14 500 | 843 |
| 8 | 29.1 | 10.5 | 12 600 | 584 |
| 9 | 30.7 | 11.6 | 15 800 | 742 |
| 10 | 25.4 | 9.4 | 14 800 | 796 |
| x | 28.5 | 10.1 | 12 200 | 431.3 |

AT RELEASE

| | | | | |
|---|---|---|---|---|
| 1 | 37.0 | 13.6 | 9 100 | 141 |
| 2 | 30.2 | 14.7 | 6 000 | 215 |
| 3 | 29.3 | 16.1 | 5 200 | 420 |
| 4 | 42.8 | 15.3 | 13 200 | 210 |
| 5 | 27.4 | 15.7 | 6 500 | 314 |
| 6 | 35.6 | 18.3 | 5 600 | 412 |
| 7 | 25.7 | 13.6 | 8 700 | 310 |
| 8 | 29.7 | 15.9 | 9 500 | 319 |
| 9 | 30.1 | 16.0 | 10 200 | 304 |
| 10 | 26.4 | 16.9 | 10 000 | 220 |
| x | 31.4 | 15.6 | 8 400 | 286.5 |

TABLE 10
EFFECT OF pPPPBs ON THE DURATION OF
THE STAY IN HOSPITAL OF PATIENTS PRESENTING
WITH A HEAD CONCUSSION

| PPPPs (days) | Placebo (days) |
|---|---|
| 17 | 30 |
| 16 | 28 |
| 17 | 18 |
| 21 | 27 |
| 13 | 28 |
| 15 | 22 |
| 18 | 42 |
| 17 | 41 |
| 17 | 31 |
| 19 | 33 |
| Median of 17 | Median of 30 |

TABLE 11
EFFECT OF pPPPBs ON THE DURATION OF THE STAY
IN HOSPITAL OF PATIENTS PRESENTING WITH AN
EXPOSED FRACTURE OF THE LONG BONES

| PPPPs (days) | Placebo (days) |
|---|---|
| 3 | 11 |
| 5 | 10 |
| 5 | 10 |
| 5 | 12 |
| 4 | 11 |
| 4 | 10 |
| 6 | 12 |
| 4 | 13 |
| 3 | 13 |
| 6 | 13 |
| Median of 4.5 | Median of 11.5 |

TABLE 12
EFFECT OF pPPPBs ON THE DURATION OF THE STAY
IN HOSPITAL FOR PATIENTS PRESENTING WITH A
PENETRATING WOUND OF THE THORAX AND/OR ABDOMEN

| PPPPs (days) | Placebo (days) |
|---|---|
| 6 | 12 |
| 6 | 17 |
| 5 | 13 |
| 11 | 18 |
| 7 | 18 |
| 10 | 16 |
| 10 | 13 |
| 8 | 15 |
| 8 | 16 |
| 9 | 12 |
| Median of 8 | Median of 15 |

TABLE 13
CHEMICAL CHARACTERIZATION OF THE PHOSPHORYLATED
POLYSUBSTITUTED POLYCARBOXYLIC PHOSPHOAMIDE
BIOPOLYMERS

| | | Isolated pPPPBs + |
|---|---|---|
| Parameter | Purified pPPPBs | calcium salts |
| % water | 7 | <5.0 |
| pH | 7.0-7.5 | 6.5-7.5 |
| % Nitrogen Content | 0.15-0.35 | 0.003-0.007 |
| % Phosphate Content | 10-15 | 34-38 |
| % Calcium Content | 1.0-2.0 | 20-24 |
| % Protein Content | 2.0-2.8 | 0.1-0.5 |
| M.W. of Proteins | 14000-16000 | n.d. |
| Isoelectric Point | 7.5-9.0 | n.d. |
| Principal Amino Acids | Lysine, | n.d. |
| | Arginine | |
| % Carbohydrate Content | 12-18 | 0.1-0.9 |

TABLE 14
HEMATOLOGICAL TOXICITY DURING CHEMOTHERAPY

| Stage of | | | | | |
|---|---|---|---|---|---|
| Chemotherapy | | | | | |
| Treatment | | | | | |
| (Type of Toxicity) | G0 | G1 | G2 | G3 | G4 |
| Hemoglobin (g/100 ml) | >11.0 | 10.9-9.5 | 9.4-8.0 | 7.9-6.5 | <6.5 |
| Leukocytes (1000/ml) | >4.0 | 3.9-3.0 | 2.9-2.0 | 1.9-1.0 | <1.0 |
| Granulocytes (1000/ml) | >2.0 | 1.9-1.5 | 1.4-1.0 | 0.9-0.5 | <0.5 |
| Platelets (1000/ml) | >100 | 99-75 | 74-50 | 49-25 | <25 |

TABLE 15
HEMATOLOGICAL RESPONSE DATA IN CANCER
PATIENTS FOLLOWING FOUR CHEMOTHERAPY
TREATMENTS IN CONJUNCTION WITH FOUR
pPPPB TREATMENTS

| Measurement | Average (cells/ml) |
|---|---|
| Hemoglobin | 11.67 |
| Hematocrit | 34.94 |
| Erythrocytes (rbc's) | 4.51 |
| Platelets | 295, |
| | 270 |
| Leukocytes | 7.0 |
| Neutrophils | 63.51 |
| Lymphocytes | 25.43 |
| Monocytes | 2.44 |
| Eosinophils | 1.61 |
| Basophils | 0.41 |

TABLE 16
HEMATOLOGICAL RESPONSE DATA IN CANCER
PATIENTS FOLLOWING RADICAL SURGERY
AND TREATMENT WITH pPPPBs

| Measurement Following Surgery | Average (cells/ml) |
|---|---|
| hemoglobin | 11.44 |
| platelets | 298, |
| | 960 |
| leukocytes | 6.52 |
| neutrophils | 66.86 |

The embodiments of the invention in which an exclusive property or privilege is claimed are defined as follows:

1. A biopolymer that is a polycarboxylic phosphoamide polysubstituted with glycopolypeptides and/or polysaccharides, wherein said biopolymer is obtained from yeast cells which have been treated with one or more stressors.

2. A biopolymer as in Formula 1, 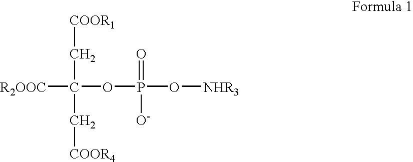
wherein R₁, R₂, R₃ and R₄ are selected from the group comprising a hydrogen, a glycopolypeptide, a polysaccharide, a branched glycopolypeptide, and a branched polysaccharide, wherein said biopolymer comprises up to four glycopolypeptides in total, four polysaccharides in total, or any combination of glycopolypeptides and/or polysaccharides totaling four, and wherein the biopolymer must contain at least one glycopolypeptide or at least one polysaccharide moiety.

3. A biopolymer derived from yeast cells comprising polycarboxylic phosphoamide polysubstituted with glycopolypeptides and polysaccharides having the formula:
[(C₆H₅O₁₀NP)-(glycopolypeptide)_{X}-(polysaccharide)_{Y}]
wherein, x equals 0-4; y equals 0-4, and wherein the sum of x plus y is less than or equal to 4 and greater than or equal to 1.

4. A phosphorylated derivative of a biopolymer derived from yeast cells comprising polycarboxylic phosphoamide polysubstituted with glycopolypeptides and polysaccharides having the formula:
[(C₆H₅O₁₀NP)-(glycopolypeptide)_{X}-(polysaccharide)_{Y}]-[(PO₄)_{z}]
wherein, z indicates a ratio of phosphate groups to biopolymer such that the weight of phosphate groups constitutes less than or equal to 3% of the total weight of the final phosphorylated biopolymer; x equals 0-4; y equals 0-4, and wherein the sum of x plus y is less than or equal to 4 and greater than or equal to 1.

5. A phosphorylated derivative of the biopolymer according to any one of claims 1, 2 or 3.

6. The biopolymer according to any one of claims 1, 2 or 3 or the phosphorylated derivative according to claim 4 or 5, wherein the molecular weights of said glycopolypeptides range from 14 to 16 kDa.

7. The biopolymer according to any one of claims 1, 2 or 3 or the phosphorylated derivative according to claim 4 or 5, wherein said polysaccharides comprise 12 to 18% of the carbohydrate content of said biopolymer.

8. The biopolymer according to any one of claims 1, 2 or 3 or the phosphorylated derivative according to claim 4 or 5, wherein the glycopolypeptides comprise 2.0 to 2.8% of the protein content of said biopolymer.

9. The biopolymer according to any one of claims 1, 2 or 3 or the phosphorylated derivative according to claim 4 or 5, wherein the phosphate groups linked to the polycarboxylic phosphoamide, glycopolypeptides and/or the polysaccharides comprise 3% of the total weight of said biopolymer.

10. The biopolymer according to any one of claims 1, 2 or 3 or the phosphorylated derivative according to claim 4 or 5, wherein said glycopolypeptides comprise at least 0.1% and no more than 0.5% of the total weight of said biopolymer.

11. The biopolymer according to any one of claims 1, 2 or 3 or the phosphorylated derivative according to claim 4 or 5, wherein said polysaccharides comprise at least 0.1% and no more than about 0.9% of the total weight of the biopolymer.

12. A pharmaceutical composition comprising a pharmaceutically acceptable excipient, and the phosphorylated derivative according to any one of claims 4, 5, 6, 7, 8, 9,10 or 11.

13. A method of producing the biopolymer of claim 1, 2, 3 or 6, 7, 8, 9, 10 or 11, comprising the following sequential steps:
(a) cultivating a strain of yeast cells to produce a standard stock culture;
(b) stressing a portion of said standard stock culture using an initial concentration of a first stressor molecule to produce a modified stock culture comprising yeast cells that can survive in the presence of the initial concentration of the first stressor molecule;
(c) repeating step (b) at least once using the modified stock culture in place of the standard stock culture and using a stressor molecule that is the same or different from the first stressor molecule;
(d) cultivating a portion of the modified stock culture produced in step (c) in the presence of the stressor molecules to generate a production culture; and
(e) isolating the biopolymer from said production culture.

14. The method according to claim 13, wherein step (b) is repeated at least once prior to step (c) using the modified stock culture in place of the standard stock culture and using an increased concentration of the first stressor molecule.

15. A method of producing the phosphorylated derivative of any one of claims 4-11 comprising the following sequential steps:
(a) cultivating a strain of yeast cells to produce a standard stock culture;
(b) stressing a portion of said standard stock culture using an initial concentration of a first stressor molecule to produce a modified stock culture comprising yeast cells that can survive in the presence of the initial concentration of the first stressor molecule;
(c) repeating step (b) at least once using the modified stock culture in place of the standard stock culture and using a stressor molecule that is the same or different from the first stressor molecule;
(d) cultivating a portion of the modified stock culture produced in step (c) in the presence of the stressor molecules to generate a production culture; and
(e) isolating the biopolymer from said production culture,
wherein the method additionally comprises the step of phosphorylating the biopolymer.

16. The method according to claim 15, wherein step (b) is repeated at least once prior to step (c) using the modified stock culture in place of the standard stock culture and using an increased concentration of the first stressor molecule.

17. The method according to claim 15 or 16, wherein the step of phosphorylating the biopolymer precedes step (e).

18. The method according to claim 15 or 16, wherein the step of phosphorylating the biopolymer follows step (e).

19. The method according to any one of claims 13, 14, 15, 16, 17 or 18, wherein said strain of yeast cells is from the genus Candida.

20. The method according to claim 19, wherein said strain of yeast cells is from the species Candida utilis.

21. The method according to claims 20, wherein said strain of yeast cells was deposited under ATCC number 9950.

22. The method according to any one of claims 13, 14, 15, 16, 17, 18, 19, 20 or 21, wherein said stressor is a terpene, a pine tree resin, a starch, a natural oil or an animal DNA.

23. The method according to claim 22, wherein said terpene is camphor, said pine tree resin is the resin of Pinus palustris, said starch is from milled soy beans, said natural oil is from milled castor beans, said animal DNA is extracted from the yolk of fertilized chicken eggs.

24. The method according to anyone of claims 13, 14, 15, 16, 17, 18, 19, 20, 21, 22 or 23, wherein stressing comprises selecting yeast cells that can survive in the presence of the stressor molecule using the following sequential steps:
(a) selecting a sample culture of yeast where growth is inhibited by 90% when exposed to an initial concentration of the stressor molecule;
(b) innoculating said culture of yeast in medium containing a similar concentration of the same stressor wherein yeast cell growth is inhibited by 90% and cultivating said yeast for 8 to 10 days;
(c) repeating steps (a) and (b) until yeast cell growth reaches the first metabolite stage of growth under the same inhibitory concentration of stressor;
(d) increasing the concentration of stressor;
(e) repeating steps (a) to (e) until the yeast cell strain no longer reaches the first metabolic stage of growth; and
(f) selecting the live yeast cell culture at the last concentration of stressor where yeast cell growth reached the first metabolite stage of growth for purifying the biopolymer.

25. Use of the biopolymer according to any one of claims 1, 2, 3, 6, 7, 8, 9, 10 or 11 in the manufacture of a medicament.

26. Use of the phosphorylated derivative according to any one of claims 4, 5, 6, 7, 8, 9, 10 or 11 in the manufacture of a medicament.

27. The use according to claim 25 or 26, wherein the medicament is for modulating the inflammatory response of an animal in need thereof.

28. The use according to any one of claims 25, 26 or 27, wherein the medicament is for modulating the immune response of an animal in need thereof.

29. The use according to any one of claims 24, 25, 26, 27 or 28, wherein the animal is a human.

30. The phosphorylated derivative according to any one of claims 4, 5, 6, 7, 8, 9, 10 or 11 for use in modulating the inflammatory and/or immune response in a patient in need thereof.

31. The phosphorylated derivative according to claim 30, wherein the patient has an injury.

32. The phosphorylated derivative according to claim 31, wherein the injury is a burn, a bone fracture, a wound or a head concussion.

33. The phosphorylated derivative according to claim 32, wherein the bone fracture is an exposed fracture.

34. The phosphorylated derivative according to claim 30, wherein the patient has a disease.

35. The phosphorylated derivative according to claim 34, wherein the disease is a neoplastic disease, an organic disease, an ischemic disease or acquired immunodeficiency syndrome.

36. The phosphorylated derivative according to claim 35, wherein the neoplastic disease is breast cancer or small-cell carcinoma of the lung.

37. The phosphorylated derivative according to claim 35 or 36, wherein the derivative is used following surgery to remove a tumor from the patient.

38. The phosphorylated derivative according to any one of claims 30, 31, 32, 33, 34, 35, 36 or 37, wherein the patient is immunocompromised.

39. The phosphorylated derivative according to claim 38, wherein the patient is immunocompromised as a result of surgery, chemotherapy, radiation therapy or a combination thereof.

40. The phosphorylated derivative according to any one of claims 30, 31, 32, 33, 34, 35, 36, 37, 38 or 39, wherein the derivative promotes healing in the patient.

41. The phosphorylated derivative according to any one of claims 30, 31, 32, 33, 34, 35, 36, 37, 38, 39 or 40, wherein the derivative inhibits microbial infection of the patient.

42. The phosphorylated derivative according to any one of claims 30, 31, 32, 33, 34, 35, 36, 37, 38, 39, 40 or 41, wherein the patient is a human.
